(19) United States
(12) Patent Application Publication
(10) Pub. No.: US 2011/0183434 A1
(43) Pub. Date: Jul. 28, 2011

(54) DIAGNOSTIC METHODS AND KITS USING FIBROBLAST GROWTH FACTOR-23

(76) Inventor: Myles Wolf, Aventura, FL (US)

(21) Appl. No.: 12/812,994

(22) PCT Filed: Jan. 15, 2009
(86) PCT No.: PCT/US09/00234

§ 371 Date: Jan. 28, 2011

Related U.S. Application Data
(60) Provisional application No. 61/021,765, filed on Jan. 17, 2008.
(60) Provisional application No. 61/034,279, filed on Mar. 6, 2008.

(51) Int. Cl. G01N 33/53

(52) U.S. Cl. 436/501

(57) ABSTRACT

The present invention describes the ability to identify chronic kidney disease (CKD) mortality risk in asymptomatic patients. For example, a patient having a normal glomerular filtration rate would be considered likely to have an increased mortality risk for chronic kidney disease upon the detection of an FGF-23 amino acid sequence that is above a normal level, but below CKD Stage 1 levels. Consequently, therapeutic strategies may be implements to prevent morbidity and mortality following chronic kidney disease progression. Such therapeutic strategies can involve phosphate reduction strategies (i.e., for example, reduced dietary intake of phosphorus and/or administration of phosphate binding compound). Further, kits are described providing instruction to determine a specific mortality risk based upon measured FGF-23 levels and estimated glomerular filtration rates.

### FIELD OF THE INVENTION

The present invention is related to the field of determining prognoses and treating chronic kidney disease. A prognostic biomarker, fibroblast growth factor 23 (FGF-23), is capable of predicting the risk of mortality due to chronic kidney disease (CKD) in an otherwise asymptomatic patient. Kits are described containing reagents having the ability to detect elevated FGF-23 for prognosis, diagnosis, and treatment in an asymptomatic CKD patient. The mortality risk prediction is based upon the progressive development of symptoms including, but not limited to, progressive kidney failure, left ventricular hypertrophy, coronary calcification, and/or death.

### BACKGROUND

Chronic kidney disease is a public health epidemic that affects ˜20 million people in the United States. Disturbed calcium-phosphate-parathyroid hormone (PTH)-vitamin D metabolism affects cardiovascular morbidity and mortality in patients with CKD, particularly in patients with End Stage Renal Disease (ESRD). Block et al., “Mineral metabolism, mortality, and morbidity in maintenance hemodialysis” J Am Soc Nephrol 15: 2208-2218 (2004); and Schmitt et al., Calcium, calcium regulatory hormones, and calcimimetics: Impact on cardiovascular mortality” J Am Soc Nephrol 17(Suppl 2): S78-S80 (2006). While some data suggests that higher phosphate levels are related to CKD progression, it has not been firmly established whether cardiovascular effects are related to CKD progression. Among factors that may be related to abnormal calcium-phosphate metabolism in patients with CKD, are believed to be hypocalcemia, hyperphosphatemia, secondary hyperparathyroidism, and inadequate production of the active hormonal form of vitamin D 1,25-dihydroxyvitamin D (1,25D).

There is little direct experimental or clinical evidence for a role of parathyroid hormone (PTH) in accelerating CPK progression. Ritz et al., “Role of calcium-phosphorous disorders in the progression of renal failure” Kidney Intl Suppl 99:S66-S70 (2005). Calcimimetics and parathyrodectomy, however, has recently been reported to attenuate CKD progression. Ogata et al., “Beneficial effects of calcimimetics on progression of renal failure and cardiovascular risk factors” J Am Soc Nephrol 14:959-967 (2003).

Chronic kidney disease is the most common cause of abnormal phosphorus metabolism, but the vast majority of patients with CKD has normal serum phosphate levels and thus abnormalities in phosphate reduction metabolism go undiagnosed in the early stages. Foley et al., “NHANES III: Influence of Race on GFR Thresholds and Detection of Metabolic Abnormalities” J Am Soc Nephrol 18:2575-8252 (2007). As a result, most of these patients are not treated with phosphorus reduction strategies such as dietary phosphorus restriction and dietary phosphorus binders until they develop over hyperphosphatemia (>4.6 mg/dl), which typically occurs in Stage 5, occasionally in Stage 4, and rarely, if at all, in earlier stages of CKD.

More sensitive tools to help identify which normophosphatemic patients might benefit from early phosphorus reduction strategies and titratation of these therapies would be of great benefit. Such tools are desperately needed because the small absolute increases in serum phosphate levels that were associated with mortality in large epidemiological studies limit their utility for clinical management decisions in individual patients.

What is needed in the art is an easy, sensitive, and direct test for a specific biomarker to identify asymptomatic patients (i.e., for example, normophosphatemic) at risk for mortality from abnormal phosphate metabolism in and due to CKD. Such a biomarker could be integrated into clinical practice to alter the management of asymptomatic kidney disease patients with the ultimate goal of improving long term clinical outcomes.

### SUMMARY

The present invention is related to the field of determining prognoses and treating chronic kidney disease. A prognostic biomarker, fibroblast growth factor 23 (FGF-23), is capable of predicting the risk of mortality due to chronic kidney disease (CKD) in an otherwise asymptomatic patient. Kits are described containing reagents having the ability to detect elevated FGF-23 for prognosis, diagnosis, and treatment in an asymptomatic CKD patient. The mortality risk prediction is based upon the progressive development of symptoms including, but not limited to, progressive kidney failure, left ventricular hypertrophy, coronary calcification, and/or death.

In one embodiment, the present invention contemplates a method, comprising: a) providing a biological sample obtained from an asymptomatic patient; b) measuring the amount of FGF-23 in said sample; and c) prophylatically treating said subject for kidney disease when the amount of FGF-23 is at least 50% above a reference population. In one embodiment, 50% above the reference population is at least 22.5 RU/ml. In one embodiment, 100% above the reference population is at least 30 RU/ml. In one embodiment, 150% above the reference popualtion is at least 45 RU/ml. In one embodiment, a reference population comprises asymptomatic individuals without chronic kidney disease. In one embodiment, the measuring detects an intact FGF-23 protein. In one embodiment, the measuring detects an FGF-23 fragment. In one embodiment, the fragment comprises a C-terminal fragment. In one embodiment, the patient has a glomerular filtration rate greater than 90 ml/minute. In one embodiment, the sample comprises a blood sample. In one embodiment, the sample comprises a tissue sample. In one embodiment, the treating comprises a phosphate reduction therapy.

In one embodiment, the present invention contemplates a method, comprising: a) providing: i) an asymptomatic patient having a mortality risk from developing chronic kidney disease; ii) a biological sample comprising an amino acid sequence derived from an FGF-23 protein; b) detecting said amino acid sequence having an activity ranging between approximately 30-60 RU/ml; and c) assigning a mortality risk by comparing said activity to a reference population. In one embodiment, the mortality risk is two times that of the reference population. In one embodiment, the mortality risk is three times that of the reference population. In one embodiment, the mortality risk is four times that of the reference population. In one embodiment, the mortality risk is five times that of the reference population. In one embodiment, the detecting comprises an antibody directed towards said amino acid sequence. In one embodiment, the patient has a glomerular filtration rate of at least 90 ml/minute. In one embodiment, the biological sample comprises urine. In one embodiment, the biological sample comprises blood plasma. In one embodiment, the biological sample comprises blood serum. In one embodiment, the biological sample comprises whole blood. In one embodiment, the biological sample comprises a tissue sample. In one embodiment, mortality risk levels are elevated when measured serum phosphate levels are between approximately 2.5-4.6 mg/dl.

In one embodiment, the present invention contemplates a method, comprising: a) providing: i) an asymptomatic patient having a mortality risk due to chronic kidney disease; ii) a biological sample comprising an oligonucleotide encoding an amino acid sequence derived from an FGF-23 protein; b) detecting said amino acid sequence having an activity ranging between approximately 30-60 RU/ml and c) assigning a mortality risk by comparing said activity to a reference population. In one embodiment, the mortality risk is two times that of the reference population. In one embodiment, the mortality risk is three times that of the reference population. In one embodiment, the mortality risk is four times that of the reference population. In one embodiment, the mortality risk is five times that of the reference population. In one embodiment, the detecting comprises an antibody directed towards said amino acid sequence. In one embodiment, the oligonucleotide comprises messenger RNA. In one embodiment, the oligonucleotide comprises DNA. In one embodiment, the detecting comprises Southern Blot analysis. In one embodiment, the detection comprises Northern Blot analysis. In one embodiment, the patient has a glomerular filtration rate of at least 90 ml/minute. In one embodiment, the patient has a glomerular filtration rate of at least 75 ml/minute. In one embodiment, the patient has a glomerular filtration rate of at least 50 ml/minute. In one embodiment, the patient has a glomerular filtration rate of at least 25 ml/minute. In one embodiment, the patient has a glomerular filtration rate of at least 10 ml/minute. In one embodiment, the biological sample comprises urine. In one embodiment, the biological sample comprises blood plasma. In one embodiment, the biological sample comprises blood serum. In one embodiment, the biological sample comprises whole blood. In one embodiment, the biological sample comprises a tissue sample. In one embodiment, mortality risk levels are elevated when measured serum phosphate levels are between approximately 2.5-4.6 mg/dl.

In one embodiment, the present invention contemplates a method, comprising: a) providing: i) an asymptomatic patient having a mortality risk due to chronic kidney disease as identified by an amino acid derived from an FGF-23 protein at levels between approximately 30-60 RU/ml; ii) a phosphate reduction therapy, wherein said therapy is capable of reducing said FGF-23 levels; b) administering said phosphate reduction therapy under conditions such that said FGF-23 levels are reduced. In one embodiment, the phosphate reduction therapy comprises a decrease in dietary phosphorus intake. In one embodiment, the phosphate reduction therapy comprises sevelamer. In one embodiment, the phosphate reduction therapy comprises lanthanum carbonate. In one embodiment, the phosphate reduction therapy comprises calcium carbonate. In one embodiment, the phosphate reduction therapy comprises calcium acetate. In one embodiment, the phosphate reduction therapy comprises alpharen.

In one embodiment, the present invention contemplates a method, comprising: a) providing: i) an asymptomatic patient having a mortality risk due to chronic kidney disease as identified by an oligonucleotide encoding an amino acid derived from an FGF-23 protein at levels between approximately 30-60 RU/ml; ii) a phosphate reduction therapy, wherein said therapy is capable of reducing said FGF-23 levels; b) administering said phosphate reduction therapy under conditions such that said FGF-23 levels are reduced. In one embodiment, the phosphate reduction therapy comprises a decrease in dietary phosphorus intake. In one embodiment, the phosphate reduction therapy comprises sevelamer. In one embodiment, the phosphate reduction therapy comprises lanthanum carbonate. In one embodiment, the phosphate reduction therapy comprises calcium carbonate. In one embodiment, the phosphate reduction therapy comprises calcium acetate. In one embodiment, the phosphate reduction therapy comprises alpharen.

In one embodiment, the present invention contemplates a kit, comprising: a) a first reagent capable of detecting an amino acid sequence derived from an FGF-23 protein; b) a control sample, wherein said sample does not contain said amino acid sequence; and, c) instructions for detecting said amino acid sequence in a biological sample under conditions such that a chronic kidney disease mortality risk is identified. In one embodiment, the instructions identify said mortality risk as associated with renal failure. In one embodiment, the instructions identify said mortality risk as associated with left ventricular hypertrophy. In one embodiment, the instructions identify said mortality risk as associated with coronary vascular calcification.

In one embodiment, the present invention contemplates a kit, comprising: a) a first reagent capable of detecting an oligonucleotide encoding an amino acid sequence derived from an FGF-23 protein; b) a control sample, wherein said sample does not contain said amino acid sequence; and, c) instructions for detecting said amino acid sequence in a biological sample under conditions such that a chronic kidney disease mortality risk is identified. In one embodiment, the instructions identify said mortality risk as associated with renal failure. In one embodiment, the instructions identify said mortality risk is associated with left ventricular hypertrophy. In one embodiment, the instructions identify said mortality risk as associated with coronary vascular calcification.

In one embodiment, the present invention contemplates a method, comprising: a) providing; i) a patient expressing at least one symptom of a kidney disease, wherein said disease confers a mortality risk on said patient; ii) a biological sample obtained from said patient, wherein said sample is suspected of comprising FGF-23; and, b) detecting said FGF-23 in said sample under conditions such that said mortality risk is identified.

In one embodiment, the present invention contemplates a kit, comprising: a) a first reagent capable of detecting FGF-23; b) a control sample, wherein said sample does not contain FGF-23; and, c) instructions for detecting FGF-23 in a biological sample under conditions such that a mortality risk conferred by a disease is identified. In one embodiment, the disease is selected from the group consisting of kidney disease and cardiovascular disease.

In one embodiment, the present invention contemplates a method, comprising: a) providing; i) a patient expressing at least one symptom of a cardiovascular disease, wherein said disease confers a mortality risk on said patient; ii) a biological sample obtained from said patient, wherein said sample is suspected of comprising FGF-23; and, b) detecting said FGF-23 in said sample under conditions such that said mortality risk is identified. In one embodiment, the cardiovascular disease is selected from the group consisting of left ventricular hypertrophy and vascular calcification.

In one embodiment, the present invention contemplates a method, comprising: a) providing; i) a patient expressing at least one symptom of a disease, wherein said patient has a compromised glomerular filtration rate; ii) a biological sample obtained from said patient, wherein said sample is suspected of comprising FGF-23; iii) a phosphate reduction composition capable of preventing hyperphosphatemia; b) detecting said FGF-23 in said sample, wherein said FGF-23 is at least 50% above a reference population; and, c) treating said patient with said phosphate reduction composition under conditions such that said hyperphosphatemia is prevented. In one embodiment, the compromised glomerular filtration rate is at least 90 ml/minute. In one embodiment, the compromised glomerular filtration rate is at least 75 ml/minute. In one embodiment, the compromised glomerular filtration rate is at least 50 ml/minute. In one embodiment, the compromised glomerular filtration rate is at least 25 ml/minute. In one embodiment, the compromised glomerular filtration rate is at least 10 ml/minute.

In one embodiment, the present invention contemplates a biomarker comprising an amino acid sequence derived from an FGF-23 protein, wherein said biomarker provides a prognosis of a chronic kidney disease mortality risk in an asymptomatic subject. In one embodiment, the amino acid sequence comprises an intact FGF-23 protein. In one embodiment, the amino acid sequence comprises an FGF-23 fragment. In one embodiment, the fragment comprises a C-terminal fragment. In one embodiment, the patient has a glomerular filtration rate greater than 90 ml/minute. In one embodiment, the mortality risk is associated with renal failure. In one embodiment, the mortality risk is associated with left ventricular hypertrophy. In one embodiment, the mortality risk is associated with coronary vascular calcification.

In one embodiment, the present invention contemplates a biomarker comprising an oligonucleotide encoding an amino acid sequence derived from an FGF-23 protein, wherein said biomarker provides a prognosis of a chronic kidney disease mortality risk in an asymptomatic subject. In one embodiment, the oligonucleotide comprises messenger RNA. In one embodiment, the oligonucleotide comprises DNA. In one embodiment, the amino acid sequence comprises an intact FGF-23 protein. In one embodiment, the amino acid sequence comprises an FGF-23 fragment. In one embodiment, the fragment comprises a C-terminal fragment. In one embodiment, the patient has a glomerular filtration rate greater than 90 ml/minute. In one embodiment, the mortality risk is associated with renal failure. In one embodiment, the mortality risk is associated with left ventricular hypertrophy. In one embodiment, the mortality risk is associated with coronary vascular calcification.

In one embodiment, the present invention contemplates a method, comprising: a) providing; i) a predialysis patient comprising an elevated FGF-23 plasma level and a normal estimated glomerular filtration rate, wherein said patient is suspected of developing chronic kidney disease; ii) a phosphate binding compound capable of reducing said FGF-23 levels; b) administering said phosphate binding compound to said patient under conditions such that said FGF-23 levels are reduced, thereby delaying the development of said chronic kidney disease. In one embodiment, the normal estimated glomerular filtration rate is at least at least 110 ml/min per 1.73 m². In one embodiment, the delayed chronic kidney disease development decreases the mortality risk of said patient.

In one embodiment, the present invention contemplates a method, comprising: a) providing; i) a predialysis patient, wherein said patient comprises an elevated FGF-23 plasma level, an estimated glomerular filtration rate greater than 60 ml/min per 1.73 m², and a renal parameter selected from the group consisting of a normal urine protein level and a normal urine hemoglobin level, wherein said patient is suspected of having an elevated mortality risk resulting from chronic kidney disease; ii) a phosphate binding compound capable of reducing said FGF-23 levels; and b) administering said phosphate binding compound to said patient under conditions such that said FGF-23 levels are reduced, thereby decreasing said mortality risk. In one embodiment, the decreased mortality risk comprises a delayed chronic kidney disease development.

In one embodiment, the present invention contemplates a method, comprising: a) providing a patient comprising an elevated FGF-23 plasma level and a renal parameter; and b) calculating a chronic kidney disease diagnostic index based upon said FGF-23 level and said second renal parameter. In one embodiment, the renal parameter is selected from the group consisting of a normal estimated glomerular filtration rate, a normal urinary protein level, and a normal urine hemoglobin level.

In one embodiment, the present invention contemplates a method, comprising: a) providing; i) a subject comprising an FGF-23 level and a renal parameter; ii) a chronic kidney disease (CKD) diagnostic index comprising a calculation of said FGF-23 level and said second renal parameter level; and b) calculating said CKD diagnostic index under conditions such that CKD is diagnosed. In one embodiment, the renal parameter comprises a normal eGFR. In one embodiment, the renal parameter comprises a normal urinary protein level. In one embodiment, the renal parameter comprises a normal hemoglobin level.

In one embodiment, the present invention contemplates a method comprising: a) providing; i) at least a first chronic kidney disease (CKD) diagnostic index calculated as an FGF-23 plasma level to renal parameter ratio; and ii) a control diagnostic index calculated as a control FGF-23 level and a control renal parameter ratio; and b) comparing said CKD diagnostic index to said control diagnostic index under conditions such that chronic kidney disease is diagnosed. In one embodiment, the comparing identifies that said CKD diagnostic index is lower than said control diagnostic index. In one embodiment, the comparing identifies that said CKD diagnostic index is higher than said control diagnostic index. In one embodiment, the renal parameter is selected from the group consisting of glomerular filtration rate, serum calcitrol, and serum creatinine. In one embodiment, the control renal parameter is selected from the group consisting of control glomerular filtration rate, control serum calcitrol, and control creatinine. In one embodiment, the method further comprises a second CKD diagnostic index. In one embodiment, the method further comprises a third CKD diagnostic index.

In one embodiment, the present invention contemplates a method, comprising: a) providing a sample derived from a subject comprising an elevated FGF-23 level and a renal parameter; b) calculating said CKD diagnostic index comprising a ratio of said FGF-23 level to said renal parameter. In one embodiment, the renal parameter is selected from the group consisting of glomerular filtration rate, serum calcitrol, and serum creatinine.

### Definitions

The term “biomarker” as used herein, refers to any biological compound related to the progressive development of chronic kidney disease. For example, a biomarker may comprise an amino acid sequence and/or a nucleic acid sequence (i.e., for example, a sequence related to FGF-23).

The term “prognosis” as used herein, refers to a medical conclusion based upon an analysis any biomarker that provides information regarding the progression of medical conditions including, but not limited to, chronic kidney disease or cardiovascular disease. Such information includes, but is not limited to the determination of a mortality risk.

The term “predicting” as used herein, refers to a method of forming a prognosis, wherein a medically trained person analyzes biomarker information.

The term “chronic kidney disease” as used herein, refers to a medical condition wherein exemplary symptoms may include, but are not limited to, hyperphosphatemia (i.e., for example, >4.6 mg/dl) or low glomerular filtration rates (i.e., for example, <90 ml/minute per 1.73 m² of body surface). However, many CKD patients may have normal serum phosphate levels in conjunction with a sustained reduction in glomerular filtration rate for 3 or more months, or a normal GFR in conjunction with sustained evidence of a structural abnormality of the kidney. In some cases, patients diagnosed with chronic kidney disease are placed on hemodialysis to maintain normal blood homeostasis (i.e., for example, urea or phosphate levels). Alternatively, “chronic kidney disease” refers to a medical condition wherein a patients has either i) a sustained reduction in GFR <60 mi/min per 1.73 m² of body surface for 3 or more months; or ii) a structural or functional abnormality of renal function for 3 or more months even in the absence of a reduced GFR. Structural or anatomical abnormalities of the kidney could be defined as but not limited to persistent microalbuminuria or proteinuria or hematuria or presence of renal cysts.

The term “diagnostic index” as used herein, refers to any calculation utilizing at least an FGF-23 level and a renal parameter to provide a quatitative evaluation of a patient's kidney function.

The term “renal parameter” as used herein refers to any physiological or biochemical component known to be indicative of renal function. For example, renal parameters may include, but are not limited to, estimated glomerular filtration rate, urine protein levels, urine hemoglobin levels, serum/urine calcium levels, serum/urine calcitrol levels, serum/urine phosphate levels, serum/urine potassium levels, or serum/urine sodium levels.

The term “mortality risk” as used herein, refers to a statistical method of forming a prognosis of survival for chronic kidney disease patients. In some cases, a mortality risk is based upon measurement of a biological compound (i.e., for example, an FGF-23 amino acid sequence and/or FGF-23 nucleic acid sequence). A mortality risk may range from between twice to ten times that when compared to a reference population. For example: i) a patient having an FGF-23 protein level of 15-20 RU/ml and a glomerular filtration rate of 120-90 ml/minute might have a mortality risk that is twice that of a reference population; a patient having an FGF-23 protein level of 26-40 RU/ml and a glomerular filtration rate of 120-90 ml/minute might have a mortality risk that is three times that of a reference population; iii) a patient having an FGF-23 protein level of 41-55 RU/ml and a glomerular filtration rate of 120-90 ml/minute might have a mortality risk that is four times that of a reference population; and iv) a patient having an FGF-23 protein level of between 56-75 RU/ml and a glomerular filtration rate of between 120-90 ml/minute might have a mortality risk that is five time that of a reference population.

The term “asymptomatic” as used herein, refers to a patient and/or subject that does not have CKD, wherein a CKD symptom includes having a reduced glomerular filtration rate (i.e., for example, between approximately 70-89 ml/min per 1.73 m² of body surface) for less than three months.

The term “predialysis” as used herein, refers to any patient and/or subject having sufficient homeostatic balance wherein all blood and/or urine parameters (i.e., for example, sodium, potassium, creatinine, urea, calcium, phosphate etc.) are within normal ranges.

The term “normal eGRF” as used herein, refers to an estimated glomerular filtration rate ranging between approximately 90-120 ml/min per 1.73 m².

The term “normal urine protein” as used herein, refers to the excretion of protein in urine ranging between approximately 0-8 mg/dl.

The term “normal urine hemoglobin” as used herein, refers to the lack of hemoglobin excretion into the urine.

The term “reference population” as used herein, refers to an individual (or patient) who does not meet criteria for chronic kidney disease as defined above. For example, FGF-23 plasma levels would be expected to range between approximately 5-20 RU/ml (i.e., for example, an average FGF-23 value would be 15 RU/ml) and glomerular filtration rates would be expected to range between approximately 95-120 ml/min per 1.73 m² of body surface). FGF-23 values above a reference population can then be determined as a percentage value (i.e., for example, 50% above the reference population would be approximately 22.5 RU/ml; 100% above the reference population would be approximately 30 RU/ml; 150% above the reference population would be approximately 45 RU/ml etc.).

The term “fragment” as used herein, when referring to an amino acid sequence, comprises at least five amino acid residues. For example, an FGF-23 protein fragment may comprise the first five amino acid residues of the C-terminal end.

The term “fragment” as used herein, when referring to an oligonucleotide sequence, comprises at least fifteen nucleic acid residues. For example, an FGF-23 gene fragment may comprise the first fifteen nucleic acids of the coding region's 3′ end.

The term “intact” as used herein, when referring to an amino acid sequence, comprises a full length protein. For example, an intact FGF-23 protein comprises all amino acid residues of the wild type sequence.

The term “intact” as used herein, when referring to an oligonucleotide sequence, encodes a full length protein. For example, an intact FGF-23 oligonucleotide comprises the coding region encoding all amino acid residues of the wild type sequence.

The term “glomerular filtration rate” as used herein, refers to a measurement capable of determining kidney function (infra). In general, normal glomerular filtration rates range between approximately 120-90 ml/minute per 1.73 m² of body surface. Compromised kidney function is assumed when glomerular filtration rates are less than 90 ml/minute per 1.73 m² of body surface. Kidney failure is probable when glomerular filtration rates fall below approximately 30 ml/minute per 1.73 m² of body surface. Dialysis is frequently initiated when glomerular filtration rates fall below approximately 15 ml/minute per 1.73 m² of body surface.

The term “renal failure” as used herein, refers to any acute (sudden) or chronic loss of the ability of the kidneys to remove waste and concentrate urine without losing electrolytes.

The term “left ventricular hypertrophy” as used herein, refers to any hypertrophy of the myocardium of a ventricle. In general, hypertrophy refers to any enlargement or overgrowth of an organ or part due to an increase in size of its constituent cells, commonly leading to a loss of function for the organ. For example, such hypertrophy may be due to chronic blood pressure overload and may be manifested electrocardiographically by an increased QRS complex voltage, frequently accompanied by repolarization changes. Echocardiography is currently the most common and sensitive method to measure cardiac hypertrophy and may be useful in establising prognosis, diagnosis and treatment of disease. For example, LVH may be correlated with FGF-23 levels using echocardiographic data.

The term “vascular calcification” as used herein, refers to any process by which vascular tissue becomes hardened by a deposit of calcium salts within its substance. For example, one such vascular tissue may be coronary vascular tissue.

The term “oligonucleotide” as used herein, refers to any polymer comprising a series of nucleic acids joined by phosphodiester bonds. For example, an oligonucleotide may comprise deoxyribonucleic acids (DNA). Alternatively, an oligonucleotide may comprise ribonucleic acids (RNA).

The term “RNA” as used herein, refers to any oligonucleotide comprising the nucleic acid uracil.

The term “DNA” as used herein, refers to any oligonucleotide comprising a mixture of the nucleic acids adenine, thymidine, cytosine, and guanosine.

The term “biological sample” as used herein, refers to any substance derived from a living organism. For example, a sample may be derived from blood as a serum sample, a plasma sample, and or a whole blood sample. Alternatively, a sample may be derived from a tissue collected, for example, by a biopsy. Such a tissue sample may comprise, for example, kidney tissue, vascular tissue and/or heart tissue. A biological sample may also comprise body fluids including, but not limited to, urine, saliva, or perspiration.

The term “antibody” as used herein, refers to any protein complex having an affinity for a specific amino acid sequence (i.e., for example, an epitope) on another protein. Polyclonal antibodies may be produced by immunization, whereas monoclonal antibodies may be produced by recombinant protein expression techniques. Generally, and antibody has two heavy amino acid chains, each comprising a light amino acid chain. It is the light amino acid chain that usually confers the epitope selectivity via hypervariability regions.

The term “phosphate reduction therapy” as used herein, refers to any method and/or compound that results in a lowering of phosphate levels in the body (i.e., for example, plasma phosphate levels) or a reduction in dietary absorption of phosphate even it it does not alter the plasma or serum levels. For example, one method comprises a reduction in the dietary intake of phosphate foods. Alternatively, compounds such as sevelamer HCl, lanthanum carbonate, calcium carbonate, calcium acetate, and alpharen act directly on physiological pathways to reduce phosphate levels (i.e., for example, by chelation binding of the phosphate within the bowels to block absorption of dietary phosphate).

The term “reagent” as used herein, refers to any substance employed to produce a chemical reaction so as to detect, measure, produce, etc., other substances.

### BRIEF DESCRIPTION OF THE FIGURES

FIG. 1 presents a schematic overview showing how fibroblast growth factor 23 (FGF-23) might regulate serum phosphate levels within a narrow range despite wide fluctuation in dietary intake.

FIG. 2 presents exemplary data demonstrating a positive correlation between C-terminal FGF-23 fragments and intact FGF-23 protein levels (r=0.74, P<0.01).

FIG. 3 presents exemplary data estimating crude, case-mix-adjusted, and multivariable-adjusted odds ratio of mortality according to Quartiles of cFGF-23. Quartile 1 is the reference group in all models. Vertical lines represent 95% confidence intervals.

FIG. 4 presents exemplary data demonstrating an odds ratio of mortality according to race (Black or White) and cFGF-23 levels above or below the median (1752 RU/ml). Vertical lines represent 95% confidence intervals.

FIG. 5 presents exemplary data demonstrating iFGF-23 raw data that is consistent with the cFGF-23 data discussed herein (Tables 7-9).

FIG. 6 presents exemplary data demonstrating the relationship of serum phosphate levels and estimated GFR in pre-dialysis chronic kidney disease patients.

FIG. 7 presents exemplary data showing mortality risk in dialysis patients as determined by 1,25 Vitamin D (FIG. 7A) or 25 Vitamin D (FIG. 7B) in dialysis patients.

FIG. 8 presents an exemplary graph of a Kaplan-Meier analyses of all ArMORR subjects for an effect of phosphate binders on mortality risk.

FIG. 9 presents an exemplary graph of a Kaplan-Meier analyses of low range normophosphatemic ArMORR subjects for an effect of phosphate binders on mortality risk.

FIG. 10 presents an exemplary graph of a Kaplan-Meier analyses of high range normophosphatemic ArMORR subjects for an effect of phosphate binders on mortality risk.

FIG. 11 presents an exemplary graph of a Kaplan-Meier analyses of ArMORR subjects having slightly elevated serum phosphate for an effect of phosphate binders on mortality risk.

FIG. 12 presents an exemplary graph of a Kaplan-Meier analyses of ArMORR subjects having serum phosphate >5.5 mg/dl for an effect of phosphate binders on mortality risk.

### DETAILED DESCRIPTION OF THE INVENTION

The present invention is related to the field of determining prognoses and treating chronic kidney disease. A prognostic biomarker, fibroblast growth factor 23 (FGF-23), is capable of predicting the risk of mortality due to chronic kidney disease (CKD) in an otherwise asymptomatic patient. Kits are described containing reagents having the ability to detect elevated FGF-23 for prognosis, diagnosis, and treatment in an asymptomatic CKD patient. The mortality risk prediction is based upon the progressive development of symptoms including, but not limited to, progressive kidney failure, left ventricular hypertrophy, coronary calcification, and/or death.

CKD is a growing public health epidemic that is associated with increased mortality due to cardiovascular diseases (CVD) and renal failure. By promoting non-atherosclerotic arterial calcification and left ventricular hypertrophy (LVH), disordered phosphate metabolism has provided an accepted risk factor for CVD development and mortality in CKD patients. Most data on phosphate metabolism and outcomes come from studies of dialysis patients; prospective data are mostly lacking in pre-dialysis CKD.

Two major reasons account for the lack of prospective data analysis. First, clinical investigators have been limited in the past by the lack of a well characterized cohorts of pre-dialysis CKD patients with: i) longitudinal blood sample collections; ii) detailed measures of CVD markers; and iii) validated outcomes. Second, the compensatory physiological mechanisms of phosphorus regulation confounded research designs when studying pre-dialysis CKD patients. A biomarker of the compensatory mechanisms was desperately needed. The discovery of FGF-23 fulfills this long felt need.

In some embodiments, the present invention contemplates an FGF-23 biomarker having improved sensitivity for identifying asymptomatic patients (i.e., for example, normophosphatemic) in need of phosphorus reduction strategies. Such biomarkers are desperately needed because the small absolute increases in serum phosphate levels that were associated with mortality in large epidemiological studies limit their utility for clinical management decisions in individual patients.

### I. Kidney Disease

Many potential kidney disease patients are not treated with phosphorus reduction strategies because of compensatory homeostatic feedback mechanisms that prevent elevated phosphate levels (i.e., for example, elevated serum phosphate levels). Elevated phosphate levels are usually treated using therapy strategies, including, but not limited to, dietary phosphorus restriction and dietary phosphorus binders. Such phosphate reduction strategies have been reported to lower FGF-23 levels in a dose-response fashion. Antoniucci et al., “Dietary phosphorus regulates serum fibroblast growth factor-23 concentrations in healthy men” J Clin Endocrinol Metab 91:3144-3149 (2006); Burnett et al., “Regulation of C-terminal and intact FGF-23 by dietary phosphate in men and women” J Bone Miner Res 21:1187-96 (2006); Nagano et al., “Effect of manipulating serum phosphorus with phosphate binder on circulating PTH and FGF23 in renal failure rats” Kidney Int 69:531-537 (2006); and Koiwa et al., “Sevelamer hydrochloride and calcium bicarbonate reduce serum fibroblast growth factor 23 levels in dialysis patients” Ther Apher Dial 9:336-339 (2005).

A. Overview

The major function of the kidneys is to remove waste products and excess fluid from the body. These waste products and excess fluid are removed through the urine. The production of urine involves highly complex steps of excretion and reabsorption. This process is necessary to maintain a stable balance of body chemicals.

The critical regulation of the body's salt, potassium and acid content is performed by the kidneys. The kidneys also produce hormones that affect the function of other organs. For example, a hormone produced by the kidneys stimulates red blood cell production. Other hormones produced by the kidneys help regulate blood pressure and control calcium metabolism. The kidneys are powerful chemical factories that perform functions including, but not limited to, removal of waste products from the body, removal of drugs form the body; maintaining body fluid balance, release of blood pressure regulation hormones, production of active vitamin D, or controlling the production of red blood cells.

B. Kidney Organization and Function

Most mammals have two kidneys, each about the size of a fist, located on either side of the spine at the lowest level of the rib cage. Each kidney contains up to a million functioning units called nephrons. A nephron consists of a filtering unit of tiny blood vessels called a glomerulus attached to a tubule. When blood enters the glomerulus, it is filtered and the remaining fluid then passes along the tubule. In the tubule, chemicals and water are either added to or removed from this filtered fluid according to the body's needs, the final product being the excreted urine.

The kidneys perform a life-sustaining job of filtering and returning to the bloodstream about 200 quarts of fluid every 24 hours. About two quarts are removed from the body in the form of urine, and about 198 quarts are recovered. Formed urine can be stored in the bladder for anywhere from 1 to 8 hours.

C. Chronic Kidney Diseases

Currently diagnosis of chronic kidney disease usually involves identifying a kidney abnormality or “biomarker” in the urine and having decreased kidney function (i.e., for example, decreased glomerular filtration rate) for three months or longer. There are many causes of chronic kidney disease. For example, the kidneys may be compromised by diseases including, but not limited to, diabetes or high blood pressure. Some kidney conditions are inherited. Other kidney conditions are congenital; that is, individuals may be born with an abnormality that can compromise kidney function.

The following medical conditions are believed to compromise kidney function and/or cause kidney damage.

1. Diabetes

Diabetes is a disease in which the body does not make enough insulin or cannot use normal amounts of insulin properly. This results in a high blood sugar level, resulting in various symptoms including, dizziness, thirst, slow wound healing, fainting, coma, and death. Diabetes is the leading cause of kidney disease.

2. Hypertension

High blood pressure (also known as hypertension) is another common cause of kidney disease and other complications such as heart attacks and strokes. High blood pressure occurs when the force of blood against arterial walls increase. When high blood pressure is controlled, the risk of complications such as chronic kidney disease is decreased.

3. Glomerulonephritis

Glomerulonephritis is a disease that causes inflammation of the kidney's filtering units called glomeruli. Glomerulonephritis may happen suddenly, for example, after a strep throat infection. In some cases, glomerulonephritis is temporary and the inflammation subsides. However, the disease may develop slowly over several years and it may cause progressive loss of kidney function.

4. Polycystic Kidney Disease

Polycystic kidney disease is the most common inherited kidney disease. It is characterized by the formation of kidney cysts that enlarge over time and may cause serious kidney damage and even kidney failure. Other inherited diseases that affect the kidneys include, but are not limited to, Alport's Syndrome, primary hyperoxaluria, and cystinuria.

5. Kidney Stones

Kidney stones are very common, and when they pass, they may cause severe pain in the back and side. There are many possible causes of kidney stones, including an inherited disorder that causes too much calcium to be absorbed from foods and urinary tract infections or obstructions. Sometimes, medications and diet can help to prevent recurrent stone formation. In cases where stones are too large to pass, treatments may be done to remove the stones or break them down into small pieces that can pass out of the body.

6. Urinary Tract Infections

Urinary tract infections occur when germs enter the urinary tract and cause symptoms such as pain and/or burning during urination and more frequent need to urinate. These infections most often affect the bladder, but they sometimes spread to the kidneys, and they may cause fever and pain in the back.

7. Congenital Diseases

Congenital diseases may also affect the kidneys (i.e., for example reflux disorders). These usually involve some problem that occurs in the urinary tract when a baby is developing in its mother's womb. One of the most common occurs when a valve-like mechanism between the bladder and ureter (urine tube) fails to work properly and allows urine to back up (reflux) to the kidneys, causing infections and possible kidney damage.

8. Drugs and Toxins

Drugs and toxins can also cause kidney problems. Using large numbers of over-the-counter pain relievers for a long time may be harmful to the kidneys. Certain other medications, toxins, pesticides and “street” drugs such as heroin and crack can also cause kidney damage.

D. Chronic Kidney Disease Detection

Early detection and treatment of chronic kidney disease plays a role in preventing a progression into kidney failure. Some simple tests can be done to detect early kidney disease including, but not limited to, blood pressure measurement, testing for excess protein in the urine, and measuring blood creatinine (see, glomerular filtration rate test below). Risk factors for chronic kidney disease include, but are not limited to, increased age, diabetes, high blood pressure, family history, ethnicity (i.e., for example, African American, Hispanic American, Asians and Pacific Islander or American Indian).

E. Chronic Kidney Disease Treatment

Many kidney diseases can be treated successfully and careful control of diseases like diabetes and high blood pressure can help prevent kidney disease or keep it from getting worse. Kidney stones and urinary tract infections can usually be treated successfully. Unfortunately, the exact causes of some kidney diseases are still unknown, and specific treatments are not yet available for them. Sometimes, chronic kidney disease may progress to kidney failure, requiring dialysis or kidney transplantation. Treating high blood pressure with special medications called angiotensin converting enzyme (ACE) inhibitors often helps to slow the progression of chronic kidney disease.

F. Kidney Failure Treatment

Kidney failure may be treated with hemodialysis, peritoneal dialysis or kidney transplantation. Treatment with hemodialysis (i.e., for example, an artificial kidney) may be performed at a dialysis unit or at home. Hemodialysis treatments are usually performed three times a week. Peritoneal dialysis is generally done daily at home. Continuous Cycling Peritoneal Dialysis requires the use of a machine while Continuous Ambulatory Peritoneal Dialysis does not.

Kidney transplants have high success rates. The kidney may come from someone who died or from a living donor who may be a relative, friend or possibly a stranger, who donates a kidney to anyone in need of a transplant.

G. Kidney Disease Symptomology

Kidney disease usually affects both kidneys. If the kidneys' ability to filter the blood is seriously damaged by disease, wastes and excess fluid may build up in the body. Symptoms of kidney disease include, but are not limited to, high blood pressure, blood and/or protein in the urine, a creatinine and/or Blood Urea Nitrogen (BUN) blood test, outside the normal range, a glomerular filtration rate (GFR) less than 60, frequent, difficult and/or painful urination (particularly at night), or puffiness around eyes, and/or swelling of hands and/or feet.

H. Glomerular Filtration Rate

Glomerular filtration rate (GFR) is a renal function test. Specifically, GFR estimates how much blood passes through the tiny filters in the kidneys, called glomeruli, each minute. The test is performed by drawing blood is drawn from a vein, usually from the inside of the elbow or the back of the hand. Alternatively, a lancet may be used to puncture the skin and make it bleed. The blood collects into a small glass tube called a pipette, or onto a slide or test strip. The blood sample is then assayed for creatinine level and in combination with several other factors the glomerular filtration rate (GFR) is estimated. These other factors involved in estimating GFR differ between adults and children. For example, the formula may incorporate factors including, but not limited to, age, creatinine measurement, gender, height, race, or weight.

Creatinine is useful in estimating GFR because this compound is a metabolic end product of muscle activity. Consequently, creatinine becomes elevated in the blood under conditions of impaired kidney function.

GFR is routinely used in the diagnosis of patients suspected of having diseases including, but not limited to, chronic kidney disease, diabetes, urinary tract infections, heart disease, high blood pressure, or urinary blockage.

According to the National Kidney Foundation, normal GFR results range from 90-120 ml/min (i.e., for example, an average of 105 ml/min), however, GFR is inversely correlated with age. In general, a GFR below 60 ml/min for 3 or more months is a symptom of chronic kidney disease, whereas a GFR below 15 ml/min is a symptom of kidney failure.

It is believed that FGF-23 levels correlate inversely with GFR and that decreased GFR may be a risk factor for CKD progression, mortality, and CVD development. All analyses discussed herein account for baseline eGFR at the time when FGF-23 and other exposures are measured. For example, high FGF-23 levels in the presence of a preserved GFR (i.e., for example, at or near normal range) may be a greater risk factor than the same FGF-23 level in patients with reduced GFR. Therefore, statistical interactions between FGF-23 levels and GFR are calculated.

I. Phosphate Reduction Strategies

1. Conventional CKD Treatment

Clinical practice guidelines recommend phosphorus reduction strategies, such as phosphorus restriction or administration of dietary phosphate binders, for hyperphosphatemic CKD patients (65). Obstacles to successful management of hyperphosphatemia in chronic kidney disease include, but are not limited to, inadequate control of dietary phosphate and non-compliance with phosphate-binder therapy.

Three major classes of phosphate binders include, but are not limited to, calcium-based binders, sevelamer HCl, and lanthanum carbonate. Calcium-based binders are effective, but their potential to contribute to total body calcium overload and vascular calcification is still of long-term clinical concern. Sevelamer HCl is effective in reducing serum phosphate, has no systemic absorption, and does not increase total body calcium load. However, sevelamer HCl binds bile acids, is not an efficient phosphate binder in an acidic environment, and contributes to metabolic acidosis. Lanthanum carbonate is a potent and selective phosphate binder that retains high affinity for phosphate over a wide pH range, does not bind bile acids or contribute to metabolic acidosis, and has the potential to reduce pill burden and increase patient compliance compared with other phosphate binders. Sprague S M., “A comparative review of the efficacy and safety of established phosphate binders: calcium, sevelamer, and lanthanum carbonate” Curr Med Res Opin. Epub Nov. 7, 2007.

A relatively new phosphate binder, Alpharen (Ineos Healthcare LTD, UK) has just completed a Phase II Federal Drug Adminsitration clinical trial. ClincialTrials.gov, “Efficacy and Safety Study of Alpharen for the Reduction of High Blood Phosphate in Hemodialysis Patients”, NCT00317694 (2007). Alpharen is a phosphate binder that absorbs phosphate from food, reducing the amount that the body can absorb.

Given the potential pathogenic role of increased FGF-23 in the development of sHPT in early CKD, reducing net phosphorus absorption may also be of substantial benefit in asymptomatic CKD patients (i.e., for example, normophosphatemic) beginning in early stages when FGF-23 levels begin to rise (i.e., for example, below Stage I levels). The data presented herein linking increased FGF-23 levels to increased CVD and mortality in normophosphatemic CKD patients, suggests that FGF-23 reduction strategies could ultimately improve clinical outcomes in CKD.

In one embodiment, the present invention contemplates directly reducing increased FGF-23 levels as a complement to conventional physiological phosphate reduction strategies thereby improving the efficacy of routine clinical interventions. Combining these two strategies may be of immediate clinical relevance for the millions of asymptomatic CKD patients for whom current guidelines do not recommend phosphate reduction strategies.

Several phosphate binder compounds are FDA-approved for treatment of hyperphosphatemia during dialysis treatment but these binders are also prescribed for hyperphosphatemic pre-dialysis patients, typically those with GFR <30 ml/min. Specific types of phosphate binders have been associated with differential rates of progression of coronary artery calcification (142) and, although the data are conflicting, dietary phosphate binders may decrease mortality during dialysis treatment (139, 157).

Since phosphate binder therapy also lowers FGF-23 levels (79, 80), making prognoses and/or diagnoses using uncontrolled data (i.e., non-adjusted) could result in confounded conclusions. For example, preliminary data from CRIC (infra) reveals that 455 of 3612 (13%) patients were treated with cation binders during baseline data collection: 297 with calcium-based binders, 146 with sevelamer hydrochloride, and 12 with lanthanum carbonate. Data adjustments made for baseline use of phosphate binders will allow a determination of the impact of specific binder type. Such data adjustments may include, but are not limited to, models excluding patients treated with phosphate binders at baseline data collection, and/or considering marginal structural models to account for time-dependent initiation of phosphate binder use during follow-up data collection periods (101).

Hyperphosphatemia is a common complication of Stage 5 CKD that contributes to calcitriol deficiency, hyperparathyroidism, renal osteodystrophy and cardiovascular mortality. While many of the adverse consequences of abnormal phosphate metabolism manifest on dialysis, pathogenesis actually begins while patients remain “normophosphatemic” in earlier stages of CKD. The data presented herein demonstrate that normal serum phosphate levels are maintained in Stage 3-4 CKD by a compensatory increase in fibroblast growth factor-23 (FGF-23) secretion (along with PTH).

These data demonstrate that calcitriol deficiency at the initiation of hemodialysis is independently associated with mortality. Thus, excessive phosphorus intake leading to increased FGF-23 secretion and decreased calcitriol may represent a modifiable risk factor for mortality due to cardiovascular disease. Current national treatment guidelines recommend aggressive phosphorus reduction strategies only for hyperphosphatemic patients. While this approach benefits an estimated 400,000 hyperphosphatemic patients in the US, there are at least 8 million normophosphatemic CKD patients who are currently untreated. Reducing phosphorus intake in normophosphatemic CKD patients therefore represents an enormous opportunity to improve the public health.

In one embodiment, the present invention contemplates increased mortality among CKD patients associated with modestly increased phosphate levels while still remaining within the normal range. In one embodiment, the present invention contemplates a method comprising reducing dietary phosphorus loading in normophosphatemic Stage 3-4 CKD patients using phosphorus binders (such as lanthanum carbonate, sevelamer, calcium-based binders) dietary phosphorus restriction or a combination of the two interventions thereby decreasing FGF-23, increasing calcitriol, and decreasing PTH levels. In one embodiment, the present invention contemplates a method comprising reducing dietary phosphorus loading in asymptomatic pre-Stage 1 CKD patients using phosphorus binders (such as lanthanum carbonate, sevelamer, calcium-based binders) dietary phosphorus restriction or a combination of the two interventions thereby decreasing FGF-23, increasing calcitriol, and decreasing PTH levels.

2. Effects Of Phosphate Binders On FGF-23

As increased phosphate intake is known to increase FGF-23 levels, FGF-23 levels are reduced in response to the administration of phosphate reduction strategies including, but not limited to, dietary phosphorous restriction or phosphate binders. Although it is not necessary to understand the mechanism of an invention, it is believed that reducing phosphorus intake and/or administering phosphate binders should lead to decreased FGF-23, increased calcitriol and decreased PTH. Animal studies support this hypothesis but human studies have not been performed to specifically study CKD. Nagano et al., “Effect of manipulating serum phosphorus with phosphate binder on circulating PTH and FGF23 in renal failure rats” Kidney Int 69(3):531-537 (2006) In this study, rats were fed a diet containing adenine for 4 weeks to establish CRI. Animals were then provided either a normal diet or a diet containing 1 or 3% sevelamer for 8 weeks continuously, or intermittently with sevelamer diet or a normal diet offered for alternating 2-week periods. Adenine-treated rats developed severe CRI, with markedly elevated serum levels of phosphorus, PTH and FGF-23, and reduced levels of serum 1,25D. Continuous treatment with sevelamer suppressed these increases throughout the study period. Serum phosphorus, PTH, and FGF-23 levels decreased rapidly when sevelamer treatments commenced and recovered rapidly once they were discontinued. However, the changes in serum FGF-23 levels began after the onset of changes in serum phosphorus and PTH levels. In conclusion, circulating PTH, and FGF-23 levels can be promptly manipulated through the control of serum phosphorus levels. Moreover, phosphate-binder treatment can effectively inhibit the elevation of serum FGF-23 levels, as well as PTH levels.

These observations support the hypothesis that increased FGF-23 may be a novel and modifiable risk factor for adverse outcomes in CKD either as a biomarker or possibly through direct tissue toxicity. In one embodiment, the present invention contemplates an FGF-23 biomarker that is more sensitive than serum phosphate levels, to identify an asymptomatic CKD patient in need of a phosphate reduction strategy.

Secretion of FGF-23 by osteocytes is stimulated by phosphorus intake and its effects are to induce phosphaturia and inhibit renal 1-α-hydroxylse activity; the latter leads to decreased renal synthesis of calcitriol, the active hormonal form of vitamin D. The net effect of increased FGF-23 secretion, therefore, is maintenance of normal serum phosphate levels in the face of wide fluctuations in phosphorus intake. In Stage 3-4 CKD patients, in whom FGF-23 secretion is chronically elevated, one adverse physiological consequence of FGF-23 compensation is early calcitriol deficiency (i.e., for example, at or near Stage 3). Calcitriol deficiency is believed to release the parathyroid glands from feedback inhibition thereby leading to hyperparathyroidism. Thus, the compensatory increase in FGF-23 to maintain normal serum phosphate levels may be involved in the pathogenesis of Stage 3 CKD secondary hyperparathyroidism (sHPT). Calcitriol deficiency in CKD has long been thought to be due to insufficient renal mass limiting the kidney's 1-α-hydroxylse activity. The present invention, however, by identifying the involvement of FGF-23 strongly challenges that oversimplified paradigm. In one embodiment, the present invention contemplates a method comprising decreasing calcitriol levels in CKD patients in response to increasing FGF-23 levels. In one embodiment, increased FGF-23 levels are due to excessive dietary phosphorus intake.

J. Kidney Injury

Kidney injury may be a result of various tissue insult including, but not limited to, toxicities, trauma, fracture, inflammation, or bruising. The kidneys are located in the flank (back of the upper abdomen at either side of the spinal column). They are deep within the abdomen and are protected by the spine, lower rib cage, and the strong muscles of the back. This location protects the kidneys from many external forces. They are well-padded for a reason—kidneys are highly vascular organs, which means that they have a large blood supply. If injury occurs, severe bleeding may result.

Kidneys may be injured by damage to the blood vessels that supply or drain them. This may be in the form of insults including, but not limited to, aneurysm, arteriovenous fistula, arterial blockage, or renal vein thrombosis. The extent of bleeding depends on the location and the degree of injury. Kidneys may also bleed profusely if they are damaged centrally (on the inside)—this is a life-threatening injury. Fortunately, most kidney injuries caused by blunt trauma occur peripherally, only causing bruising of the kidney (usually a self-limiting process).

People with undiagnosed kidney conditions—such as angiomyolipoma (benign tumor), ureteropelvic junction obstruction (congenital or acquired UPJ Obstruction), and other disorders—are more susceptible to kidney injuries and more likely to have serious complications if they occur. Other causes of kidney injury and bleeding are medical procedures. Kidney biopsies, nephrostomy tube placements, or other surgeries can cause an abnormal connection between an artery and vein (arteriovenous fistula). This is usually a self-limiting problem, but close observation is usually needed. Injury to the kidney can also disrupt the urinary tract, causing leakage of the urine from the kidney.

Each kidney filters about 1700 liters of blood per day and concentrates fluid and waste products into about 1 liter of urine per day. Because of this, the kidneys receive more exposure to toxic substances in the body than almost any other organ. Therefore, they are highly susceptible to injury from toxic substances.

Analgesic nephropathy is one of the most common types of toxic damage to the kidney. Exposure to lead, cleaning products, solvents, fuels, or other nephrotoxic chemicals (those which can be toxic to the kidney) can damage kidneys. Excessive buildup of body waste products, such as uric acid (that can occur with gout or with treatment of bone marrow, lymph node, or other disorders) can also damage the kidneys.

Inflammation (irritation with swelling and presence of extra immune cells) caused by immune responses to medications, infection, or other disorders may also injure the structures of the kidney, usually causing various types of glomerulonephritis or acute tubular necrosis (tissue death). Autoimmune disorders may also damage the kidneys.

Injury to the kidney may result in short-term damage with minimal or no symptoms. Alternately, it can be life-threatening because of bleeding and associated shock, or it may result in acute renal failure or chronic renal failure.

Ureteral injuries (injuries to the tubes which carry urine from the kidneys to the bladder) can also be caused by trauma (blunt or penetrating), complications from medical procedures, and other diseases in the retroperitoneum such as retroperitoneal fibrosis (RPF), retroperitoneal sarcomas, or metastatic lymph node positive cancers.

Medical therapies (such as OB/GYN surgeries, prior radiation or chemotherapy, and previous abdominopelvic surgeries) increase the risk for ureteral injuries. Symptoms of acute kidney injury may include, but is not limited to, blood in the urine, flank pain (severe), abdominal pain, back pain, nausea, vomiting, abdominal swelling, decreased urine output, or inability to urinate. Symptoms of chronic kidney injury may include, but is not limited to, irritability, weight loss, or constipation (most likely in conjunction with a toxic injury such as lead poisoning). There may also be signs of hemorrhage and shock, including rapid heart rate and falling blood pressure. Toxic injury or injury from inflammation may cause acute or chronic renal failure. A urinalysis may show blood (i.e., for example, hematuria) and/or sediment or crystals reflective of inflammation or toxic accumulations of uric acid or other substances. A Complete Blood Count (CBC) may show bleeding, infection, or inflammation. Other blood tests may reveal toxic levels of suspected substances. An electrolyte analysis of the blood may demonstrate increased potassium, urea, or creatinine. Other tests including, but not limited to, kidney x-ray, abdominal CT scan, or abdominal MRI scan may show damage to the kidney. Alternatively, a renal scan may show problems with kidney blood flow or an angiography of the artery or vein may show occlusion of blood flow to or from the kidney. Intravenous pyelograms (IVP) may also determine kidney function, wherein an IVP may be repeated after treatment of kidney injury to assess functioning of the traumatically injured kidney.

### II. Fibroblast Growth Factor-23

Fibroblast growth factor-23 (FGF-23) is a recently discovered phosphorus regulating hormone. In patients with kidney disease, increased FGF-23 secretion helps prevent hyperphosphatemia but inhibits renal production of 1,25 dihydroxyvitamin D (1,25D). Hyperphosphatemia and low 1,25D levels were associated with mortality but the impact of FGF-23 on mortality was unknown.

FGF-23 is a 251 amino acid protein secreted by osteoblasts and osteocytes in adults (40) and other tissues during development. Benet-Pages et al., “FGF-23 is processed by proprotein convertases but not by PHEX” Bone 35:455-462 (2004); Sitara et al., “Homozygous ablation of fibroblast growth factor-23 results in hyperphosphatemia and impaired skeletogenesis, and reverses hypophosphatemia in Phex-deficient mice” Matrix Biol. 23:421-432 (2004); Quarles L. D., “FGF-23, PHEX, and MEPE regulation of phosphate homeostasis and skeletal mineralization” Am J Physiol Endocrinol Metab 285:E1-E9 (2003); Shimada et al., “Targeted ablation of FGF-23 causes hyperphosphatemia, increased 1,25 dihydroxyvitamin D levels and severe growth retardation” J Bone Miner Res. 17: 168 (2002); and Shimada et al., “Mutant FGF-23 responsible for autosomal dominant hypophosphatemic rickets is resistant to proteolytic cleavage and causes hypophosphatemia in vivo” Endocrinology 143:3179-3182 (2002).

Biologically active FGF-23 induces phosphaturia by decreasing phosphate reabsorption in the proximal tubule, and inhibits renal 1-α hydroxylase leading to decreased conversion of 25D to 1,25D (i.e., for example, the hormonal form of vitamin D). Shimada et al., “FGF-23 transgenic mice demonstrate hypophosphatemic rickets with reduced expression of sodium phosphate cotransporter type 1α” Biochem Biophys Res Commun. 314:409-414 (2004); Saito et al., “Circulating FGF-23 is regulated by 1 alpha,25-dihydroxyvitamin D3 and phosphorus in vivo” J Biol Chem. 280:2543-2549 (2005); Shimada et al., “Transgenic mice expressing FGF23 demonstrate hypophosphatemia with low serum 1,25-dihydroxyvitamin D and rickets/osteomalacia” J Bone Miner Res. 16:151 (2001); Larsson et al., “Transgenic mice expressing fibroblast growth factor 23 under the control of the alpha1(1) collagen promoter exhibit growth retardation, osteomalacia, and disturbed phosphate homeostasis” Endocrinology 145:3087-3094 (2004); Shimada et al., “FGF-23 is a potent regulator of vitamin D metabolism and phosphate homeostasis” J Bone Miner Res. 19:429-435 (2004); Saito et al., “Human fibroblast growth factor-23 mutants suppress Na⁺-dependent phosphate co-transport activity and 1alpha,25-dihydroxyvitamin D3 production” J Biol Chem. 278:2206-2211 (2003).

“Primary” syndromes of FGF-23 excess are believed marked by hypophosphatemia, renal phosphate wasting, and inappropriately low 1,25D levels for the degree of hypophosphatemia. These aspects of FGF-23 physiology were confirmed in: i) transgenic mice that overexpress FGF-23; ii) mice administered exogenous FGF-23; and iii) various human syndromes of hypophosphatemia caused by excessive FGF-23. In contrast, FGF-23 depletion such as FGF-23 null mice and patients with inactivating FGF-23 mutations, develop hyperphosphatemia with excessive 1,25D. FGF-23 is also believed involved in other rare disorders of phosphate metabolism.

Oral phosphorus loading has been observed to stimulate FGF-23 secretion, thereby inducing phosphaturia and suppressing renal 1,25D production. On the other hand, phosphorus restriction exerts the opposite effects: FGF-23 levels decrease leading to renal phosphate reabsorption and enhanced gut absorption of phosphorus due to increased 1,25D. Consequently, it has been suggested that serum phosphate levels are regulated within a narrow homeostatic range by FGF-23 to control wide fluctuations in phosphorus intake. Saito et al., “Human fibroblast growth factor-23 mutants suppress Natdependent phosphate co-transport activity and 1alpha,25-dihydroxyvitamin D3 production” J Biol Chem. 278:2206-2211 (2003); Bai et al., “The autosomal dominant hypophosphatemic rickets R176Q mutation in fibroblast growth factor 23 resists proteolytic cleavage and enhances in vivo biological potency” J Biol Chem. 278:9843-989 (2003); Riminucci et al., “FGF-23 in fibrous dysplasia of: bone and its relationship to renal phosphate wasting” J Clin Invest. 112:683-692 (2003). Anonymous, “Autosomal dominant hypophosphataemic rickets is associated with mutations in FGF23” The ADHR Consortium. Nat Genet. 26:345-348 (2000); Jonsson et al., “Fibroblast growth factor 23 in oncogenic osteomalacia and X-linked hypophosphatemia” N Engl J Med. 348:1656-1663 (2003); Yamazaki et al., “Increased circulatory level of biologically active full-length FGF-23 in patients with hypophosphatemic rickets/osteomalacia” J Clin Endocrinol Metab. 87:4957-4960 (2002); Yamamoto et al., “The role of fibroblast growth factor 23 for hypophosphatemia and abnormal regulation of vitamin D metabolism in patients with McCune-Albright syndrome” J Bone Miner Metab. 23:231-237 (2005); Ferrari et al., “Fibroblast growth factor-23 relationship to dietary phosphate and renal phosphate handling in healthy young men” J Clin Endocrinol Metab. 90:1519-1524 (2005); Antoniucci et al., “Dietary phosphorus regulates serum fibroblast growth factor-23 concentrations in healthy men” J Clin Endocrinol Metab. 91:3144-3149 (2006); Burnett et al., “Regulation of C-terminal and intact FGF-23 by dietary phosphate in men and women” J Bone Miner Res. 21:1187-96 (2006).

A. Physiological Role of FGF-23

In one embodiment, the present invention contemplates that increased FGF-23 levels in asymptomatic CKD patients (i.e., for example, normophosphatemic) are associated with kidney disease progression, left ventricular hypertrophy, coronary artery calcification, and increased mortality.

Fibroblast growth factor-23 (FGF-23) was first described as a pathogenic factor in rare hypophosphatemic syndromes in which “primary” increases in biologically active FGF-23 caused renal phosphate wasting leading to hypophosphatemia with inappropriately low levels of 1,25-dihydroxyvitamin D (1,25D), often leading to the development of rickets and/or osteomalacia. Jonsson et al., “Fibroblast growth factor 23 in oncogenic osteomalacia and X-linked hypophosphatemia” N Engl J Med 348:1656-1663 (2003); White et al., “The autosomal dominant hypophosphatemic rickets (ADHR) gene is a secreted polypeptide overexpressed by tumors that cause phosphate wasting” J Clin Endocrinol Metab 86:497-500 (2001); Autosomal Dominant Hypophosphataemic Rickets Consortium, “Autosomal dominant hypophosphataemic rickets is associated with mutations in FGF-23” Nat Genet 26:345-348 (2000); Shimada et al., “Cloning and characterization of FGF23 as a causative factor of tumor-induced osteomalacia” Proc Natl Acad Sci USA 98:6500-6505 (2001); De Beur et al., “Tumors associated with oncogenic osteomalacia express genes important in bone and mineral metabolism” J Bone Miner Res 17:1102-1110 (2002); and Berndt et al., “Phosphatonins and the regulation of phosphorus homeostasis” Am J Physiol Renal Physiol 289:F1170-1182 (2005). In contrast, FGF-23 depletion has been observed to result in hyperphosphatemia, excessive 1,25D levels, ectopic calcification, and early mortality. Shimada et al., “Targeted ablation of FGF-23 demonstrates an essential physiological role of FGF-23 in phosphate and vitamin D metabolism” J Clin Invest 113:561-568 (2004); Benet-Pages et al., “An FGF23 missense mutation causes familial tumoral calcinosis with hyperphosphatemia” Hum Mol Genet 14:385-390 (2005); and Stubbs et al., “Role of hyperphosphatemia and 1,25-dihydroxyvitamin D in vascular calcification and mortality in fibroblastic growth factor 23 null mice” J Am Soc Nephrol 18:2116-2124 (2007).

B. Kidney Disease And FGF-23

Kidney disease is commonly associated with FGF-23 excess but the causative factors are complex and have not been well understood. In patients with kidney disease, normophosphatemia may be maintained despite declining nephron mass. Although it is not necessary to understand the mechanism of an invention it is believed that normophosphatemia is maintained by progressive increases in FGF-23 levels during CKD progression. It is further believed that FGF-23 stimulates greater per-nephron phosphate excretion (in concert with increased PTH) and limits dietary phosphorus absorption by feedback inhibition of renal 1,25D synthesis. Gutierrez et al., “Fibroblast growth factor-23 mitigates hyperphosphatemia but accentuates calcitriol deficiency in chronic kidney disease” J Am Soc Nephrol 16:2205-2215 (2005).

Mortality in kidney disease has usually been associated with cardiovascular disease due to decreased 1,25D. Wolf et al., “Vitamin D levels and early mortality among incident hemodialysis patients” Kidney Int 72:1004-1013 (2007); and Ritz et al., “Skeletal abnormalities in chronic renal insufficiency before and during maintenance hemodialysis” Kidney Int 4:116-127 (1973). However, patients with kidney disease also develop symptoms that are reflective of decreased FGF-23 protein levels including, but not limited to hyperphosphatemia, ectopic calcification, and/or early mortality. Cozzolino et al., “Pathogenesis of vascular calcification in chronic kidney disease” Kidney Int 68:429-436 (2005). Thus, although increased serum phosphate and decreased 1,25D levels are associated with increased mortality, it was unknown if compensatory increases in FGF-23 secretion promote or protect against mortality. Block et al., “Mineral metabolism, mortality, and morbidity in maintenance hemodialysis” J Am Soc Nephrol 15:2208-2218 (2004).

In one study, FGF-23 levels were observed to increase in early stages of diagnosed chronic kidney disease before hyperphosphatemia development. Gutierrez et al., “Fibroblast growth factor-23 mitigates hyperphosphatemia but accentuates calcitriol deficiency in chronic kidney disease” J Am Soc Nephrol 16:2205-2215 (2005). Once hyperphosphatemic, however, patients have been identified as already having a markedly increased risk of mortality. Go et al., “Chronic kidney disease and the risks of death, cardiovascular events, and hospitalization” N Engl J Med 351:1296-1305 (2004). In a recent prospective study, FGF-23 levels were implicated as a potential causal mechanism of more rapid progression of kidney disease. Fliser et al., “Fibroblast Growth Factor 23 (FGF23) Predicts Progression of Chronic Kidney Disease: The Mild to Moderate Kidney Disease (MMKD) Study” J Am Soc Nephrol 18:2601-2608 (2007). Although the results were independent of baseline renal function and serum phosphate levels, residual confounding by these factors was cited as a potential limitation. In addition, many of the CKD patients, in association with their high FGF-23 levels, had received previous activated Vitamin D therapy, which is believed to stimulate FGF-23 secretion.

In contrast, the data presented herein, which examined a more homogenous population of incident dialysis patients free of confounding by previous activated vitamin D therapy, and in which there was a lack of inverse correlation between FGF-23 and 1,25D levels (i.e., for example, perhaps because severe renal failure overshadowed the inhibitory effect of FGF-23 on 1,25D secretion), offers additional support for a direct FGF-23 effect. Although it is not necessary to understand the mechanism of an invention, it is believed that at high concentrations FGF-23 levels (i.e., for example≧100 RU/ml), FGF-23 likely binds different FGF receptors with sufficiently high affinity (even in the absence of the co-receptor Klotho) to stimulate the production of factors such as osteopontin that have been implicated in the development of vascular disease but are normally generated in response to basic FGF. Yu et al., “Analysis of the biochemical mechanisms for the endocrine actions of fibroblast growth factor-23” Endocrinology 146:4647-4656 (2005).

Thus, while FGF-23 depletion and 1,25D intoxication are associated with mortality in the setting of normal renal function, the data presented herein suggests that FGF-23 excess and 1,25D deficiency appear to be associated with mortality in renal failure. Further, the present analysis excluded patients who were previously treated with activated vitamin D in order to minimize co-therapy confounding factors. Indeed, FGF-23 levels were much higher in previous reports of prevalent dialysis patients likely because many had received activated vitamin D. Larsson et al., “Circulating concentration of FGF-23 increases as renal function declines in patients with chronic kidney disease, but does not change in response to variation in phosphate intake in healthy volunteers. Kidney Int 64:2272-2279 (2003); and Imanishi et al., “FGF-23 in patients with end-stage renal disease on hemodialysis” Kidney Int 65:1943-1946 (2004).

1. Secondary Hyperparathyroidism

Secondary hyperparathyroidism (sHPT) is a common complication of CKD that is associated with bone disease, CVD, and mortality (63-65). Although FGF-23 and PTH levels are correlated in CKD (60, 66), and FGF receptors are expressed on parathyroid cells (67), the direct effects of FGF-23 on PTH regulation remain unclear. However, a recent study suggests that FGF-23 may provide an inhibitory influence on PTH. Ben-Dov et al., “The parathyroid is a target organ for FGF23 in rats” J Clin Invest. 117:4003-4008 (2007). Nevertheless, through its inhibition of 1,25D synthesis, compensatory increases in FGF-23 levels in early CKD have been implicated as a potential indirect mechanism of sHPT (60, 62). One small cohort study of early Stage 3 CKD patients, provided data showing significantly increased FGF-23 levels at a time when PTH levels remained normal (68). Thus, FGF-23 may be a novel, early mechanism or biomarker of sHPT that is detectable not only before changes in serum phosphate, but also before PTH levels rise.

Studies of rare forms of hypophosphatemic rickets implicated FGF-23 excess as a cause of phosphaturia and inhibition of renal 1-a hydroxylase (44, 51-56). Subsequent studies demonstrated the role of FGF-23 in normal phosphate homeostasis (57). Although it is not necessary to understand the mechanism of an invention, it is believed that increased FGF-23 levels in early CKD may maintain normophosphatemia in the setting of decreased nephron mass. It is further believed that by inhibiting 1,25D synthesis, FGF-23 contributes to the pathogenesis of sHPT in CKD.

2. Chronic Kidney Disease Progression

Increased serum phosphate levels are associated with accelerated progression of CKD (69-71), perhaps by accelerating tubulointerstitial fibrosis (72). In contrast, phosphorus restricted diets may slow kidney function decline (73). To date, one prospective study of 177 subjects suggested an association between increased FGF-23 levels and more rapid progression of CKD (74). Although the results were independent of baseline renal function and serum phosphate levels, residual confounding by these factors and by prior therapy with active vitamin D and phosphate binders were potential limitations.

C. Ethnicity

The initial FGF-23 human studies as discussed above were conducted in mostly Caucasian and Asian populations. None of these studies compared levels across races and none reported levels among Blacks and Hispanics.

Blacks and Hispanics are believed predisposed to the rapid progression of CKD and CVD development (82). Although there are known race/ethnic differences in mineral metabolism among dialysis and healthy patients (83), data are sparse regarding pre-dialysis CKD patients. For example, Blacks demonstrate decreased urinary phosphate excretion and increased serum phosphate levels compared to Caucasian despite increased PTH levels. Bell et al., “Evidence for alteration of the vitamin D-endocrine system in blacks” J Clin Inves; 76:470-473 (1985): Foley et al., “NHANES III: Influence of Race on GFR Thresholds and Detection of Metabolic Abnormalities” J Am Soc Nephrol 18:2575-8252 (2007) and Menon et al., “Relationship of phosphorus and calcium-phosphorus product with mortality in CKD” Am J Kidney Dis 46:455-463 (2005). Although it is not necessary to understand the mechanism of an invention, it is believed that decreased FGF-23 expression could account for this discrepancy. It is further believed that, along with increased PTH, decreased FGF-23 levels could also account for higher 1,25D levels among Blacks despite their lower 25D substrate than Caucasian.

In the data presented herein, Blacks demonstrated decreased urinary phosphate excretion and increased serum phosphate levels compared to Caucasians despite increased PTH. In addition, despite significantly decreased 25D stores, Blacks had significantly increased 1,25D levels (83).

Although it is not necessary to understand the mechanism of an invention, it is believed that decreased FGF-23 levels among Blacks may play a role in the higher serum phosphate and lower urinary phosphate (despite high PTH), and higher 1,25D levels (despite lower 25D stores) in Blacks as compared to other races. Until the present invention, human studies comparing FGF-23 levels across races, and specifically among Blacks and Hispanics, was not available (54, 58-60, 62). In the data presented herein, a FGF-23 levels were lower by approximately 22-33% in both Blacks and Hispanics as compared to Caucasians.

These data suggest that decreased FGF-23 levels among Blacks and Hispanics could represent a physiological adaptation to maintain normal bone mineralization and circulating 1,25D levels in the face of high rates of vitamin D deficiency and sHPT. These observations also suggest that decreased FGF-23 might contribute to the well known, but poorly understood, survival advantage of Blacks on dialysis. Supporting this possibility is that Blacks had significantly lower FGF-23 levels than Caucasians overall. Among patients with lower FGF-23 levels, Blacks demonstrated significantly decreased risk of mortality compared to Caucasian. Further studies should assess racial/ethnic differences in FGF-23 physiology and its impact on clinical outcomes across the spectrum of kidney disease and in health.

D. Phosphorous Homeostasis And FGF-23

Hyperphosphatemia is believed to be a risk factor for cardiovascular disease (CVD), kidney disease progression, and mortality in chronic kidney disease (CKD). Even subtle increases in serum phosphate levels within the normal range are independently associated with adverse outcomes, both in CKD and non-CKD populations. However, hyperphosphatemia is uncommon in pre-Stage 1 CKD patients (i.e., for example, those not yet undergoing dialysis) and the small differences in serum phosphate levels that were associated with poor outcomes in large CKD cohorts provide limited their utility for clinical management decisions in individual patients. Superior biomarkers (i.e., for example, those which are more sensitive than phosphate levels) are desperately needed to focus therapies for disordered phosphate metabolism earlier in CKD. In one embodiment, the present invention contemplates FGF-23 as one such biomarker.

Fibroblast growth factor-23 (FGF-23) is a recently discovered hormone secreted by osteoblasts and osteocytes that regulates phosphorus homeostasis and vitamin D metabolism. Progressive increases in FGF-23 levels beginning in early CKD help maintain normophosphatemia in the face of declining nephron mass and thus, increased FGF-23 levels are detectable long before hyperphosphatemia first appears.

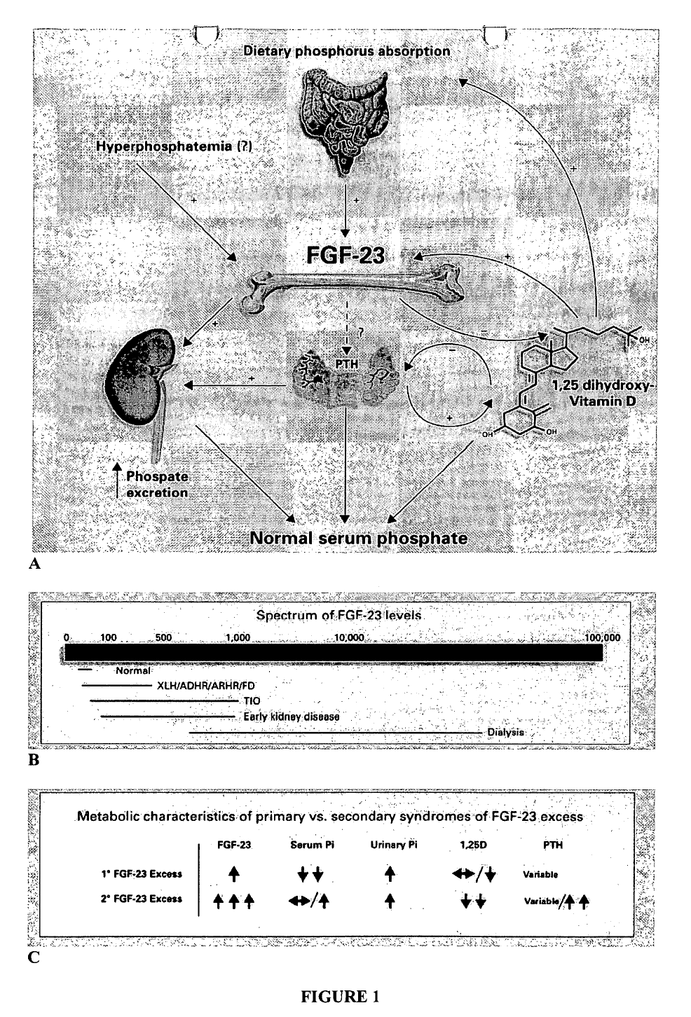

Although it is not necessary to understand the mechanism of an invention it is believed that FGF-23 regulation of phosphorous homeostasis may occur via a series of classic negative endocrine feedback loops involving 1,25-dihydroxyvitamin D (1,25D), urinary phosphate excretion, and dietary phosphorus absorption. For example, FGF-23 secretion by osteoblasts and osteocytes may be stimulated (indicated by +) by factors including, but not limited to: i) increased dietary phosphorus intake; ii) exposure to 1,25D; or increased serum phosphate levels. On the other hand, FGF-23 secretion may be inhibited (indicated by −) by factors including, but not limited to: i) low dietary phosphorus intake; ii) hypophosphatemia; or low 1,25D levels. See, FIG. 1A.

It has been observed that FGF-23 protein binds to a FGF receptor having an optimized affinity in the presence of a co-receptor, termed Klotho. Data collected in the renal proximal tubule demonstrates that increased FGF-23 protein binding stimulates urinary phosphate excretion by down-regulating expression of a luminal sodium-phosphate cotransporter (i.e., for example, NaPᵢ₋₂ₐ and NaP_{i-2c}.

In addition, FGF-23 protein may inhibit 25-hydroxyvitamin D-1-β-hydroxylase leading to decreased circulating levels of 1,25D. Decreased 1,25D levels, in turn, are believed to lower gut phosphorus absorption and release the parathyroid glands from feedback inhibition, thereby increasing circulating parathyroid hormone (PTH) levels, which further augment urinary phosphate excretion.

Presumed direct effects of FGF-23 protein on the parathyroid glands, bone mineralization and other organs are less clear. A spectrum of FGF-23 levels can be observed under normal conditions and in a variety of syndromes of FGF-23 excess. For example, circulating FGF-23 levels are 10- to 20-fold above normal range (i.e., for example, ˜30-60 RU/ml using a C-terminal FGF-23 fragment assay) in patients with hereditary hypophosphatemic rickets syndromes, including X-linked hypophosphatemia (XLH), autosomal dominant hypophosphatemic rickets (ADHR), autosomal recessive hypophosphatemic rickets (ARHR), and fibrous dysplasia (FD). See, FIG. 1B. While these data suggest that FGF-23 levels were often even higher in patients with tumor-induced osteomalacia (TIO), the highest levels were encountered in patients with kidney disease (especially those on dialysis) where concentrations between approximately 1,000-50,000 fold were seen above the normal range.

Different ‘signature patterns’ of FGF-23, serum Pᵢ, urinary Pᵢ, 1,25D, and PTH levels may differentiate “primary” FGF-23 excess syndromes (i.e., for example, hereditary diseases and TIO) versus “secondary” FGF-23 excess syndromes (i.e., for example, chronic kidney disease). See, FIG. 1C. In general, high FGF-23 protein levels and normal-to-high serum phosphate (Pᵢ) levels are seen in patients with kidney disease, as compared to hypophosphatemia, which is generally seen in hereditary syndromes. In general, 1,25D levels tend to be lower and PTH levels higher in patients with kidney disease as compared with the hereditary syndromes. Urinary fractional excretion of phosphate (FEₚₒ₄) is high in both pre-dialysis kidney disease and genetic hypophosphatemic disorders.

The data presented herein assesses the mortality risk according to Quartiles of baseline serum phosphate levels in a prospective study comprising a cohort of 10,044 incident hemodialysis patients. Baseline FGF-23 levels versus mortality risk was analyzed in a nested case-control sample (i.e., from non-diseased patients). Two hundred (200) randomly selected subjects who subsequently died (cases) and two hundred (200) randomly selected subjects who survived (controls) the first year of dialysis were included in the study. These subjects included fifty 50 case subjects and 50 control subjects selected from each Quartile of baseline serum phosphate measurements. In one embodiment, the present invention contemplates that increased FGF-23 levels is predictive of kidney disease mortality in all Quartiles of baseline serum phosphate levels.

Serum phosphate levels in the highest phosphate Quartile (i.e., for example, >5.5 mg/dl) were associated with a modest 20% multivariable-adjusted increased risk of mortality compared to normal levels (HR 1.2; 95% CI 1.1, 1.4). In the nested sample, median C-terminal FGF-23 protein fragment levels were significantly higher in Case subjects as compared to Control subjects (2260 vs. 1406 RU/ml; P<0.01). The differences were greatest in Quartiles 1-3 (i.e., for example, phosphate levels <5.5 mg/dl).

In a multivariable-adjusted analyses, patients were stratified into FGF-23 Quartiles according to increasing FGF-23 levels. The data analysis showed that FGF-23 level increases, expressed in ascending Quartiles was associated with a monotonic increased risk of mortality. The data analysis was performed by: i) a continuous scale (i.e., for example, OR/unit increase in log FGF-23 1.8; 95% CI 1.4, 2.4); or ii) by individual FGF-23 Quartiles (Quartile [Q]1: reference; Q2: OR 1.6, 95% CI 0.8, 3.3; Q3: 4.5, 95% CI 2.2, 9.4; Q4 5.7, 95% CI 2.6, 12.6). The results were virtually unchanged using either a C-terminal FGF-23 assay or an intact FGF-23 assay.

The FGF-23 Quartile statistical analysis revealed that increased FGF-23 protein levels are associated with increased mortality among incident hemodialysis patients independent of serum phosphate levels and other risk factors. In one embodiment, the present invention contemplates a method for identifying a need for phosphorus and/or FGF-23 reduction therapy by detecting elevated FGF-23 protein levels in an asymptomatic patient. In one embodiment, the present invention contemplates a method for selecting an asymptomatic patient at risk for developing Stage 1 chronic kidney disease (i.e., for example, normophosphatemic) for treatment with a phosphate reduction strategy.

In one embodiment, the present invention contemplates a method of determining mortality risk in a pre-dialysis patient comprising measuring an FGF-23 protein level. In one embodiment, the FGF-23 protein level is measured in the urine. In one embodiment, the FGF-23 protein level is measured in blood plasma. In one embodiment, the FGF-23 protein level is measured in blood serum. In one embodiment, the FGF-23 protein level is measured in whole blood. In one embodiment, mortality risk levels are elevated when measured FGF-23 levels are between approximately 20-80 RU/ml. In some embodiment, mortality risk levels are elevated when measured FGF-23 levels are between approximately 80-600 RU/ml. In one embodiment, mortality risk levels are elevated when measured FGF-23 levels are greater than 600 RU/ml. In one embodiment, mortality risk levels are elevated when measured serum phosphate levels are between approximately 2.5-4.6 mg/dl in conjunction with FGF-23 levels that are at least 50% higher than a reference population.

Analysis of the FGF-23 Quartile data presented herein shows a strong “dose-response”- type linear relationship when assessing the relationship between FGF-23 protein levels and chronic kidney disease mortality risk. For example, each ascending Quartile (i.e., Q1-Q4) of baseline FGF-23 protein measurement was associated with a relative increase in chronic kidney disease mortality risk. A similar assessment of chronic kidney disease-related mortality risk associated with increasing baseline phosphorous level Quartiles had a substantially smaller predictive value than when using baseline FGF-23 level Quartiles. In one embodiment, the present invention contemplates a method for treating an asymptomatic kidney disease subject with a phosphorous reduction compound.

The results presented herein are relevant to accepted CKD clinical practice. The 20% increased risk of mortality was observed only among members in the uppermost phosphate Quartile versus the lowest phosphate Quartile. This observation is comparable to previous reports. Block et al., Mineral metabolism, mortality, and morbidity in maintenance hemodialysis” J Am Soc Nephrol 15:2208-2218 (2004). Similarly, serum phosphate levels in excess of 3.5 mg/dl were associated with ˜28% increased risk of mortality in studies of pre-dialysis kidney disease patients and in non-kidney disease populations. Kestenbaum et al., “Serum phosphate levels and mortality risk among people with chronic kidney disease” J Am Soc Nephrol 16:520-528 (2005); and Tonelli et al., “Relation between serum phosphate level and cardiovascular event rate in people with coronary disease” Circulation 112:2627-2633 (2005).

In contrast, FGF-23 was shown to be a CKD biomarker having vast superiority over phosphate, by markedly improved sensitivity (i.e., for example, an approximate 30-fold increase in sensitivity). The data presented herein revealed a monotonic, “dose-response” type relationship between FGF-23 and mortality such that patients in the highest FGF-23 Quartile were at a nearly 600% multivariable-adjusted increased mortality risk as compared to those with the lowest level Quartiles.

In addition, the association between FGF-23 and chronic kidney disease mortality was strongest when analyzing the phosphate Quartiles Q1-Q3. Although it is not necessary to understand the mechanism of an invention, it is believed that these results suggest that serum phosphate measurements, which are influenced by dietary absorption, urinary and dialysis clearance, bone mineralization, and soft tissue deposition, provide only a partial assessment of risk associated with abnormal phosphorus metabolism, especially when serum phosphate levels are normal. In comparison, FGF-23 levels were most informative when serum phosphate levels were relatively low. While overt hyperphosphatemia is a risk factor for mortality, FGF-23 measurements could represent a novel biomarker to assess risk in the absence of hyperphosphatemia.

### III. Chronic Kidney Disease (CKD) and Cardiovascular Disease (CVD)

CKD is a growing public health epidemic that affects −12% of the US population, or −20 million people. Coresh et al., “Chronic kidney disease awareness, prevalence, and trends among U.S. adults, 1999 to 2000” J Am Soc Nephrol. 1.16:180-188 (2005). Although the overwhelming economic and medical impact of the growing dialysis population (Stage 5 CKD: >300,000) is widely recognized, >8,000,000 people suffer from CKD Stages 3 or 4, which have been suggested as risk factors for CVD mortality. Muntner et al., “Renal insufficiency and subsequent death resulting from cardiovascular disease in the United States” J Am Soc Nephrol. 13:745-753 (2002); Sarnak et al., “Kidney disease as a risk factor for development of cardiovascular disease: a statement from the American Heart Association Councils on Kidney in Cardiovascular Disease, High Blood Pressure Research, Clinical Cardiology, and Epidemiology and Prevention” Circulation 108:2154-2169 (2003); Morion et al., “The epidemiology of chronic kidney disease stages 1 to 4 and cardiovascular disease: a high-risk combination” Am J Kidney Dis. 45:223-232 (2005); and Foley et al., “Epidemiology of cardiovascular disease in chronic renal disease” J Am Soc Nephrol; 9:16-23 (1998).

CVD and CKD are believed to share common risk factors. For example, the presence of CVD is associated with progressive CKD, de novo and recurrent CVD events are more common in all stages of CKD, and the clinical outcomes are far worse in CKD patients than in the general population. Elsayed et al., “Cardiovascular Disease and Subsequent Kidney Disease” Arch Intern Med. 167:1130-1136 (2007); Go et al., “Chronic kidney disease and the risks of death, cardiovascular events, and hospitalization” N Engl J Med. 351:1296-1305 (2004); and Weiner et al., “Chronic kidney disease as a risk factor for cardiovascular disease and all-cause mortality: a pooled analysis of community-based studies” J Am Soc Nephrol. 15:1307-1315 (2004). As a result of these “disease multiplying” interrelationships, Stage 3-4 CKD patients' mortality risk due to CVD is significantly greater than their risk of progressing to renal failure. Shulman et al., “Prognostic value of serum creatinine and effect of treatment of hypertension on renal function. Results from the hypertension detection and follow-up program. The Hypertension Detection and Follow-up Program Cooperative Group” Hypertension 13:180-193 (1989); and Keith et al., “Longitudinal follow-up and outcomes among a population with chronic kidney disease in a large managed care organization” Arch Intern Med. 164:659-663 (2004).

Elevated serum phosphate levels, even within the normal range, are associated with LVH, diastolic dysfunction, and increased CVD-related mortality in patients with CKD (37). In addition, high phosphate concentrations promote non-atherosclerotic arterial calcification by stimulating metaplasia of vascular smooth muscle cells into an osteogenic phenotype (11).

Although FGF-23 is associated with disordered phosphate metabolism, there are virtually no data on FGF-23 and surrogate markers of CVD such as LVH and arterial calcification. Although it is not necessary to understand the mechanism of an invention, it is believed that that increased FGF-23 may be associated with LVH and coronary calcification in pre-dialysis CKD, acting as a sensitive marker of net phosphorus exposure or perhaps through direct toxicity on the cardiovascular system. Alternatively, since FGF-23 is secreted by osteocytes, it could act as a novel marker of total vascular calcification in which high FGF-23 could reflect excess expression by vascular “bone.”

A. Patterns of Vascular and Cardiac Injury in CKD: Role of Phosphate

Traditional risk factors for atherosclerosis do not fully account for the extent of vascular disease in CKD, suggesting that additional, CKD-specific risk factors also contribute.

Furthermore, in addition to typical atherosclerosis, CKD patients also develop extensive non-atherosclerotic calcification of the arterial media, an uncommon phenotype outside CKD. Cozzolino et al., “Pathogenesis of vascular calcification in chronic kidney disease” Kidney Int. 68:429-436 (2005). Indeed, young CKD patients with few traditional risk factors have been reported to develop extensive vascular calcification. Goodman et al., “Coronary-artery calcification in young adults with end-stage renal disease who are undergoing dialysis” N Engl J Med. 342:1478-1483 (2000). Several other studies have demonstrated an association between increased arterial calcification and mortality in dialysis patients. Blacher et al., “Arterial calcifications, arterial stiffness, and cardiovascular risk in end-stage renal disease” Hypertension 38:938-942 (2001); Moo et al., “Natural history of vascular calcification in dialysis and transplant patients” Nephrol Dial Transplant. 19:2387-2393 (2004).

In addition, validated surrogate measures of vascular calcification, including rapid aortic pulse wave velocity and increased pulse pressure which reflect decreased vascular compliance due to circumferential calcification, were associated with CKD mortality. Pannier et al., “Stiffness of capacitive and conduit arteries: prognostic significance for end-stage renal disease patients” Hypertension 45:592-596 (2005); Covic et al., “Aortic pulse wave velocity and arterial wave reflections predict the extent and severity of coronary artery disease in chronic kidney disease patients” J Nephrol. 18:388-396 (2005); Guerin et al., “Arterial stiffening and vascular calcifications in end-stage renal disease” Nephrol Dial Transplant 15:1014-1021 (2000).

B. Left Ventricular Hypertrophy

In one embodiment, the present invention contemplates an association between FGF-23 levels and left ventricular hypertrophy (LVH).

CKD is believed associated with high rates of left ventricular hypertrophy (LVH) such that congestive heart failure accounts for at least 50% of CVD-related deaths on dialysis. LVH is an independent risk factor for heart failure and mortality in virtually all populations, including non-CKD, dialysis, and pre-dialysis CKD. The prevalence of LVH increases with each stage of progressive CKD, and up to 75% of incident dialysis patients are affected. While hypertension and volume overload contribute to LVH in CKD, normotensive dialysis patients also manifest LVH, suggesting additional mechanisms. Levin A., “Clinical epidemiology of cardiovascular disease in chronic kidney disease prior to dialysis” Semin Dial. 16:101-105 (2003); Levy et al., “Prognostic implications of echocardiographically determined left ventricular mass in the Framingham Heart Study” N Engl J Med. 322:1561-1566 (1990); Silberberg et al., “Impact of left ventricular hypertrophy on survival in end-stage renal disease” Kidney Int. 36:286-290 (1989); Foley et al., “Clinical and echocardiographic disease in patients starting end-stage renal disease therapy” Kidney Int. 47:186-192 (1995); Meeus et al., “Pathophysiology of cardiovascular disease in hemodialysis patients” Kidney Int 76:140-147 (2000); and Strozecki et al., “Parathormon, calcium, phosphorus, and left ventricular structure and function in normotensive hemodialysis patients” Ren Fail 23:115-26 (2001).

Due to its effects on vascular compliance, arterial calcification is a CKD-specific risk factor for LVH. For example, rats that underwent parathyroidectomy, nephrectomy, and were administered physiological doses of PTH and a high phosphorus diet developed hyperphosphatemia and LVH without vascular calcification. Neves et al., “Adverse effects of hyperphosphatemia on myocardial hypertrophy, renal function, and bone in rats with renal failure” Kidney Int. 66:2237-22344 (2004). Furthermore, correction of hyperphosphatemia with daily hemodialysis was independently associated with regression on LVH. Ayus et al., “Effects of short daily versus conventional hemodialysis on left ventricular hypertrophy and inflammatory markers: a prospective, controlled study” J Am Soc Nephrol. 16:2778-88 (2005); and Achinger et al., “Left Ventricular Hypertrophy: Is Hyperphosphatemia among Dialysis Patients a Risk Factor?” J Am Soc Nephrol 17:255-261 (2006).

Transthoracic echocardiography TTE) is a non-invasive test that is used in clinical practice to evaluate cardiac structure (chamber dimensions and volumes, wall thickness, valves) and systolic (ejection fraction) and diastolic function (ratio of early to late transmittal velocities (E-to-A ratio) by Doppler), among other data. LVH can be assessed by TTE, defined as a left ventricular mass index (LVMI)≧134 g/m² in men and≧110 g/m² in women. Verdecchia et al., “Prognostic value of left ventricular mass and geometry in systemic hypertension with left ventricular hypertrophy” Am J Cardiol. 78:197-202 (1996). Risk factors for LVH in CKD are believed to include, but not limited to: i) the severity of GFR reduction (135); ii) increased blood pressure; and iii) anemia. Levin et al., “Prevalent left ventricular hypertrophy in the predialysis population: identifying opportunities for intervention” Am J Kidney Dis. 27:347-354 (1996); and Levin et al., “Left ventricular mass index increase in early renal disease: impact of decline in hemoglobin” Am J Kidney Dis. 34:125-34 (1999). Although abnormalities in mineral metabolism are also independently associated with LVH, these studies did not examine FGF-23. Strozecki et al., “Parathormon, calcium, phosphorus, and left ventricular structure and function in normotensive hemodialysis patients” Ren Fail 23:115-126 (2001); Ernesto et al., “Left Ventricular Hypertrophy in Nondiabetic Predialysis CKD” Am J Kidney Dis. 46:320-327 (2005)

C. Vascular Calcification

In one embodiment, the present invention contemplates an association between FGF-23 levels and vascular calcification. In one embodiment, the vascular calcification comprises coronary vessel calcification.

Vascular calcification is believed to be an organized cellular process that begins in early, CKD and is mediated, in part, by up-regulation of osteoblast factors in vascular smooth muscle cells exposed to high phosphate concentrations. Goodman et al., “Vascular calcification in chronic kidney disease” Am J Kidney Dis. 43:572-579 (2004); Giachelli et al., “Vascular calcification and inorganic phosphate” Am J Kidney Dis. 38:34-37 (2001); Giachelli et al., “Vascular calcification mechanisms” J Am Soc Nephrol 15:2959-2964 (2004); Kramer et al., “Association between chronic kidney disease and coronary artery calcification: the Dallas Heart Study” J Am Soc Nephrol. 16:507-513 (2005); Sigrist et al., “Vascular calcification and cardiovascular function in chronic kidney disease” Nephrol Dial Transplant. 21:707-714 (2006); Jono et al., “Phosphate regulation of vascular smooth muscle cell calcification” Circ Res. 87:E10-E7 (2000) Chen et al., “Phosphorus and uremic serum up-regulate osteopontin expression in vascular smooth muscle cells” Kidney Int. 62:1724-31 (2002); and Moe et al., “Uremia induces the osteoblast differentiation factor Cbfal in human blood vessels” Kidney Int. 63:1003-1011 (2003).

While numerous population and laboratory studies examined the role of phosphate in vascular calcification, there are no published studies of FGF-23 and vascular calcification in pre-dialysis CKD. Jono et al., “Phosphate regulation of vascular smooth muscle cell calcification” Circ Res. 87:E10-E7 (2000); and Chen et al., “Phosphorus and uremic serum up-regulate osteopontin expression in vascular smooth muscle cells” Kidney Int. 62:1724-31 (2002); and Moe et al., “Uremia induces the osteoblast differentiation factor Cbfal in human blood vessels” Kidney Int. 63:1003-1011 (2003). Indeed, non-atherosclerotic arterial calcification is believed to result from metaplasia of vascular smooth muscle cells into osteoblasts, and FGF-23 is secreted by osteoblasts.

Although it is not necessary to understand the mechanism of an invention, it is believed that increased FGF-23 levels in CKD could reflect auxiliary production by vascular “bone”. It is further believed that marked increases in FGF-23 concentrations are directly toxic. Indeed, at the high FGF-23 concentrations observed in CKD, FGF-23 likely binds different FGF receptors with sufficiently high affinity, even in the absence of its co-receptor Klotho (77). Such altered binding patterns may stimulate the production of factors such as osteopontin that have been implicated in the development of CVD but are normally generated in response to basic FGF (78).

Calcification of the coronary arteries is a highly prevalent and rapidly progressive form of vascular injury in dialysis and pre-dialysis CKD patients that is associated with increased mortality (138, 139). It often develops in young patients with CKD in whom the prevalence of traditional risk factors for atherosclerosis are low, suggesting the importance of other mechanisms (140). Abnormal mineral metabolism is now viewed as a mechanism of CAC in CKD patients, based on both large observational and interventional studies and supportive in vitro and animal work (141-143). Electron beam CT (EBCT) provides a noninvasive and quantitative measurement of CAC that has been validated as an independent risk factor for future cardiovascular events in the general population and in CKD patients (144-145). EBCT obtains 30-40 thin slice tomographs at a rapid speed that reduces motion artifact (146). Multi-detector computed tomography (MDCT) uses multiple scanners with short rotation times and allows for acquisition of high-quality images that are comparable to those obtained with EBCT (147). The CAC score is based on Hounsfield units in the artery wall and the area of calcium deposits (121). While EBCT adds to the predictive capacity of the Framingham risk score (146), and CAC scores predict coronary artery disease in the general population with 95-99% sensitivity and 66-77% specificity (148, 149), there is less data in pre-dialysis CKD and no published studies relating CAC to FGF-23 levels.

While CAC scores provide a precise measure of coronary artery calcification, extensive calcification throughout the large conduit arteries, including the aorta, carotids, iliofemorals, etc, is common in CKD patients (132). When there is significant calcification of peripheral vasculature, arterial stiffness increases and compliance decreases. This can manifest as increased pulse pressure, which is associated with increased mortality on dialysis (150).

An additional measure of global arterial stiffness is the aortic pulse wave velocity, which can be measured non-invasively by recording the arterial pulses at proximal and distal points and measuring the velocity of flow to and from the heart (132). With increased stiffness and hence increased calcification, the velocity of reflection of the systolic pulse wave from the periphery back to the ascending aorta increases (the forward pulse hits the peripheral resistance earlier, namely in the stiff conduit arteries). Aortic pulse wave velocity has been used in a variety of studies in CKD in which it correlated with CAC scores and was independently associated with CVD and mortality (130-131).

In one study, a pulse wave velocity of 10.75 m/s predicted future CVD-related mortality with 84% sensitivity and 73% specificity. Pannier et al., “Stiffness of capacitive and conduit arteries: prognostic significance for end-stage renal disease patients” Hypertension 45:592-596 (2005). Although several studies have examined pulse wave velocity and serum phosphate, calcium and vitamin D levels, none examined a relationship with FGF-23 levels.

Ankle-brachial index (ABI) is a measure of lower extremity peripheral vascular disease, that is also independently associated with CVD events and mortality (151). The ABI reflects the ratio of systolic BP in the posterior tibial or the dorsalis pedis arteries to that in the brachial artery measured by ultrasonography (152). A low ABI (<0.9) is 95% sensitive and 100% specific for angiographically documented lower extremity arterial disease (129) and is predictive of CVD in the general population (151) and in CKD (153). A high ABI (>1.3) reflects poorly compressible arteries that are seen with medial artery calcification (154), and is associated with hyperparathyroidism and increased mortality in CKD (155, 156). Currently, there are no reported studies comparing ABI and FGF-23.

VI. The Chronic Renal Insufficiency Cohort (CRIC) Analysis

In one embodiment, the present invention contemplates that FGF-23 represents a novel risk factor for CVD and kidney disease progression, acting either as a biomarker of disordered phosphate metabolism or through direct toxicity at the tissue level.

FGF-23 levels are expected to be measured at 2 separate time points in ˜3,800 participants in the racially and ethnically diverse Chronic Renal Insufficiency Cohort (“CRIC”), the largest and most detailed prospective study of pre-dialysis CKD in the US (125).

CRIC offers a unique opportunity to efficiently test some embodiments as contemplated by the present invention. The CRIC provides the opportunity to measuring baseline and follow-up FGF-23 levels from stored samples derived from an ethnically diverse population of over 3800 CKD patients. In one embodiment, using the CRIC establishes FGF-23 as a novel biomarker to predict many adverse outcomes in CKD patients. In one embodiment, the biomarker provides a justification to administer interventional phosphorus reduction strategies to asymptomatic CKD patients who would otherwise not be considered.

In some embodiments, the present invention contemplates associations between increased FGF-23 levels and factors including, but not limited to; i) biomarkers of mineral metabolism: serum and urinary phosphate; calcium, PTH; dietary phosphorus intake; ii) measures of CVD structure and function: left ventricular hypertrophy and diastolic dysfunction by echocardiography; coronary artery calcification by electron beam computerized tomography (EBCT); vascular compliance by aortic pulse wave velocity; peripheral vascular disease by ankle brachial index (ABI). In some embodiments clinical end-points will be measured including, but not limited to: i) renal: progression of renal disease assessed by slope of change in GFR; time to 50% reduction in GFR; and time to end stage renal disease requiring dialysis or transplantation; ii) CVD: new CVD events such as new-onset of myocardial infarction, angina, coronary artery revascularization, peripheral vascular disease, and stroke; iii) hospitalizations: all-cause and due to CVD; and iv) mortality: all-cause and due to CVD.

A. The CRIC Study Population 1. Overview

The CRIC Study was established in 2003 to prospectively examine risk factors for progression of renal disease and the development of CVD in a large, racially and ethnically diverse, nationally representative cohort of CKD patients. The CRIC database contains detailed demographic, socioeconomic and nutritional data, repeated measures of renal and cardiovascular function, and validated outcomes, including hospitalizations, progression to dialysis, major CVD events and mortality. Blood and urine specimens are collected annually during the follow-up period and stored for future prospective cohort and nested case-control or case-cohort studies.

2. Patient Recruiting, Enrollment and Follow-Up

Seven recruiting sites from across the US recruit a nationally representative cohort of 3800 subjects: University of Pennsylvania, Johns Hopkins University/University of Maryland, Case Western Reserve University, University of Michigan at Ann Arbor, University of Illinois at Chicago, Tulane University Health Science Center, and Kaiser Permanente of Northern California/University of California at San Francisco. The enrollment strategy for CRIC targets specific distributions of age, sex, race and diabetes in order to support a stratified analyses (i.e., for example, 50% of subjects may be aged 45-64, and 25% may be 21-44 and 65-74; 50% women, 50% diabetes, 40% Caucasian, 40% Black, 20% Other).

3. Characteristics of CRIC Participants

To date, CRIC and the ancillary Hispanic CRIC extension has enrolled −3,753 subjects. Baseline characteristics of the first 3,612 participants in the entire cohort and those in the ¹²⁵1-iothalamate/EBCT subcohort are similar. The age distribution is representative of national data for CKD (128). Blacks are at significantly increased risk of developing progressive CKD and were therefore over sampled in CRIC (46% of the cohort). This maximizes power for detailed analyses stratified by race. The ancillary Hispanic CRIC study will ensure a final set of 380 Hispanic subjects, or, 10% of the population. Detailed data on socioeconomic status are collected, including annual household income and highest level of education achieved. These will serve as critical covariates for analyses of race and outcomes in which lower socioeconomic: status could be a vital confounder.

Hypertension and diabetes mellitus are highly prevalent at enrollment with rates, that mirror national averages for CKD populations (128). Prior history of CVD is present in up to 22% of the cohort in the form of coronary artery disease, coronary revascularization, myocardial infarction, congestive heart failure, and peripheral vascular disease. Reflecting the recruiting strategy, the majority of subjects have Stage 3 CKD with a mean eGFR of 43 ml/min. The distribution of GFR in CRIC is ideal for our proposal given that serum phosphate levels remain normal in the vast majority of Stage 3 patients (81) while their FGF-23 levels begin to rise (60) and the incidence rates of sHPT (81), major CVD events, and mortality increase dramatically (7). Indeed, further analysis of baseline mean serum phosphate levels in CRIC yields the following percentile stratification (n=3546):

| Phosphate centiles | mg/dl |
|---|---|
| 25^{th} percentile | 3.3 |
| 50^{th} percentile | 3.7 |
| 75^{th} percentile | 4.1 |
| 90^{th} percentile | 4.5 |

Thus, over 90% of all subjects had normal serum phosphate levels (<4.6 mg/dl) at enrollment. Even among patients with the most severe reduction in eGFR at enrollment (<30 ml/min), mean serum phosphate levels were 4.1±0.8, remaining in the normal range. This illustrates the capacity of renal compensation to maintain normophosphatemia in the face of severe kidney disease and strongly supports the need for novel biomarkers of disordered phosphate metabolism with greater resolution than the highly regulated serum phosphate level.

B. CRIC Analysis Procedures

After written informed consent is obtained, extensive clinical evaluations are performed at baseline, during annual clinic visits, and via telephone interview at six-month intervals.

1. Screening Visits

At the initial screening visit, eligibility is assessed and written informed consent is obtained. Demographic data and non-fasting blood specimens are collected to further determine eligibility and calculate eGFR. Urine specimens are obtained and tested for glucose, protein, and blood.

2. Baseline Visit

Subjects who are eligible return within three months for their baseline visit, which is considered the date of enrollment. Data are collected including, but not limited to: i) detailed medical history and comprehensive medication list: name, dose, and frequency; ii) blood pressure: using standard sphygmomanometers; mean levels of 3 readings at rest while seated; iii) anthropometric measures: i.e., for example, height, weight, mid-abdominal circumference, bioelectrical impedance iv) questionnaires: i.e., for example, dietary intake, physical activity, quality of life, depression, cognitive function; v) fasting blood specimens for tests including, but not limited to: complete blood count: i.e., for example, hemoglobin, hematocrit, white blood cells, platelets, comprehensive metabolic panel: i.e., for example, sodium, potassium, chloride, bicarbonate, urea nitrogen, creatinine, glucose, iPTH, calcium, phosphate, magnesium, total protein, albumin, bilirubin, alkaline phosphatase, AST, ALT, complete cholesterol panel, triglycerides, cystatin C, hemoglobin ArC, homocysteine, troponin I, fibrinogen, and uric acid; vi) Additional plasma and serum samples may be stored frozen for future uses; vii) spot and 24-hour urine samples for assay of creatinine, protein, albumin and urea nitrogen; 100 cc of urine are stored for future use, such as for urinary phosphate measurements that we will perform; viii) a specimen for DNA extraction and storage is collected upon consent; ix) iothalamate GFR subcohort (n=1,300) patients undergo a baseline test, which will be repeated 2 and 4 years after enrollment using standard techniques (126, 127); x) cardiovascular measures: i.e., for example, electrocardiography, ankle-brachial index (ABI): a validated measure of peripheral arterial occlusive disease (129), Aortic pulse wave velocity: a validated measure of vascular compliance that is directly correlated with vascular calcification and associated with mortality in prospective studies (15-17, 130, 131).

3. Annual Follow-Up Visits

Up to six years of annual longitudinal follow-up visits are expected depending on the date of enrollment. The following data are obtained including, but not limited to: i) questionnaires, i.e., for example, nutritional intake; ii) fasting blood and urine specimens: all subjects provide serum, plasma and “clean catch” urine specimens at each of the annual visits for repeat testing of the above parameters; iii) factors related to disordered phosphorus metabolism including but not limited to, serum phosphate, calcium, PTH, etc; iv) additional plasma, serum and urine samples are collected and stored for future assays, such as repeated FGF-23 and urinary phosphate measurements; v) cardiovascular measures: i.e., for example, electrocardiography, ankle-brachial index, aortic pulse wave velocity (132), echocardiography, including measures of left ventricular mass index (LVMI), systolic and diastolic function, and valvular disease; vi) the iothalamate GFR subcohort will undergo electron beam computerized tomography (EBCT) to assess coronary artery calcification.

4. Exposures

The primary exposures are circulating levels of FGF-23 and serum phosphate levels. It is expected that FGF-23 levels will be measured in all participants at two time points: at year 1 and 2 after enrollment. Serum phosphate levels are measured annually on an ongoing basis as part of CRIC. There are several reasons for the schedule of FGF-23 measurements. First, the timing of the initial FGF-23 measurement coincides with the baseline echocardiogram, EBCT, and iothalamate GFR, which will allow initial cross-sectional studies of FGF-23 levels and the primary surrogate measures of CVD, namely LVH and coronary artery calcification with precise measures of iothalamate GFR in many subjects. Concomitant measures of the primary exposures and outcomes at baseline will also strengthen the prospective analyses of FGF-23 and CVD markers by providing repeated measures. Second, substantially less demand for CRIC samples at the year 1 and year 2 visits compared to baseline ensures adequate sample volume for FGF-23 measurements in all subjects. Justification for repeated rather than isolated measurements of the primary exposures.

Several studies demonstrated an increased risk of mortality on dialysis or in pre-dialysis CKD associated with increased serum phosphate levels. While these studies were instrumental in the development of practice guidelines that emphasize serum phosphate reduction, they focused only on isolated measures at a single time point (63-65). Subsequent studies in dialysis patients using time-varying analyses with repeated measures of serum phosphate levels yielded optimal target ranges for serum phosphate levels (in terms of mortality) that were different than the previous fixed covariate analyses (113). Similar analyses involving repeated measures over time have not been performed in pre-dialysis CKD. This is a major limitation because serum phosphate levels vary with time due to changes in diet, concurrent illness, and therapy with vitamin D or phosphate binders, etc.

While serum phosphate control has been an accepted clinical practice, the present invention contemplates that a reduction of net phosphorus exposure may be guided by FGF-23 levels and may be a prognostic indicator in early CKD patients with normal serum phosphate levels. A prospective study using repeated measures of phosphate and FGF-23 for the first time and link them to CVD and renal surrogates and hard clinical end-points in pre-dialysis CKD.

C. Data Analysis

1. One Year Preliminary Data

a. Reported Cardiovascular Disease and Kidney Disease End-Points

Among the initial 1493 subjects who have completed year of follow-up, 101 developed outpatient diagnoses of new-onset CVD: 44 acute myocardial infarctions, 34 strokes, and 23 cases of atrial fibrillation. A total of 1,030 hospitalizations were reported over the same period including 43 for acute myocardial infarction, 135 for angina, 77 for congestive heart failure, 22 for coronary artery bypass grafting, 59 for arrhythmia, 36 for stroke, 26 for transient ischemic attack, 32 for peripheral vascular disease, 9 for carotid artery disease and 2 for amputation. In contrast, only 9 hospitalizations were for progression to end stage renal disease: 7 for kidney transplantation and 2 for initiation of dialysis. The markedly greater rates of CVD events compared with progression to renal failure support the critical need for studies of mechanisms of CVD in CKD and the large numbers of events will provide excellent power to test our hypotheses.

b. Mortality

There were 39 deaths among the 1493 subjects followed for 1 year, or 26 deaths/1000 person-years of follow-up. Conservatively assuming −10% loss to follow-up during the first year (it was actually 6%), :3500 participants will have 3500 person years of follow up and thus, we expect 91 deaths during the first year. Given up to 6 years of follow-up in many subjects, we expect at least 400 500 deaths based on these initial results. This is a conservative estimate because it is based on a constant mortality over time but in actuality, mortality rates in CKD increase as renal function declines (7).

2. Secondary Hyperparathyroidism

Secondary hyperparathyroidism (sHPT) is a common complication of CKD that is associated with bone disease and CVD (65). PTH levels typically begin to rise in stage 3 CKD, and the incidence of abnormally high PTH levels (>65 pg/ml) increases beginning in late stage 3-early stage 4 and continues to rise as CKD progresses (65). Although it is not necessary to understand the mechanism of an invention, it is believed that FGF-23 mediated inhibition of renal 1,25D synthesis, begins earlier in CKD and predisposes the patient to the development of sHPT. Data may suggest that increased baseline FGF-23 levels predict the development of new-onset increases in PTH to >65 pg/ml and whether the magnitude of increased FGF-23 is associated with the subsequent rate of change over time in PTH levels on a continuous scale in all subjects. Currently, there are no published studies of longitudinal measures of PTH or FGF-23 over time in pre-dialysis CKD.

3. Kidney Disease Progression

Increased FGF-23 levels may be associated with progression of kidney disease, either as a marker of excessive phosphorus intake or via direct renal toxicity (i.e., for example, perhaps by promoting progression of tubulointerstitial fibrosis). The risk of adverse renal outcomes as related to FGF-23 can be studied by using calculations including, but not limited to; i) a slope change in GFR over time on a continuous scale; ii) time to 50% reduction in GFR; and iii) time to end stage renal disease requiring dialysis or transplantation. For GFR-based analyses, eGFR and cystatin C will be examined in the entire cohort and ¹²⁵I-iothalamate clearance in a subcohort.

4. Markers of Cardiovascular Disease

Validated surrogate markers of adverse CVD outcomes will be examined. Further, in later years hard clinical end-points of CVD will be assessed.

CVD surrogates examined include, but are not limited to, i) left ventricular mass/hypertrophy (at year 1 and 4); ii) coronary artery calcification (year 1 and 4); iii) aortic pulse wave velocity (biannually); and iv) ankle-brachial index (annually). In some embodiments, the cardiovascular surrogate markers include, but are not limited to, left ventricular hypertrophy, coronary artery calcification (CAC), or aortic pulse wave velocity.

Hard clinical end-points include, but are not limited to, major CVD events, hospitalizations (all-cause and CVD-related), and all-cause and CVD mortality. All hard clinical end-points are initially ascertained by patient self-report (or family and primary physician in the event of death) and then medically validated. Sources to be used to confirm events/outcomes include, but are not limited to. review of hospital charts and discharge summaries, confirmation with primary care physicians, acquisition of death certificates, and review of claims databases and death registries.

### IV. Kits

In another embodiment, the present invention contemplates kits for the practice of the methods of this invention. The kits may include one or more containers containing an amino acid and/or an oligonucleotide detection method of this invention. The kit can optionally include a non-diseased cell culture to be utilized as a control. The kit can optionally include nucleic acids capable of hybridizing to an FGF-23 gene region (i.e., for example, PCR primers). The kit can optionally include enzymes capable of performing PCR (i.e., for example, DNA polymerase, Taq polymerase and/or restriction enzymes). The kit can optionally include a pharmaceutically acceptable excipient and/or a delivery vehicle (e.g., a liposome). The reagents may be provided suspended in the excipient and/or delivery vehicle or may be provided as a separate component which can be later combined with the excipient and/or delivery vehicle. The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in the diagnosis, detection, and/or treatment of kidney disease within a mammal. In particular the disease can include any one or more of the disorders described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### V. Detection Methodologies

A. Detection of RNA

In some embodiments, the present invention contemplates detecting expressed mRNA in a biological sample (i.e., for example, a blood sample). mRNA expression may be measured by any suitable method, including but not limited to, those disclosed below.

In some embodiments, RNA is detection by Northern blot analysis. Northern blot analysis involves the separation of RNA and hybridization of a complementary labeled probe. In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes. In still further embodiments, RNA (or corresponding cDNA) is detected by hybridization to a oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

B. Detection of Protein

In other embodiments, gene expression may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

C. Remote Detection Systems

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personal and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of a virus infection) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### Experimental

### EXAMPLE I

### Correlational Analysis Showing Relationship Of FGF-23 and Mortality due to Kidney Disease

### Methods

The present study obtained data from the Accelerated Mortality on Renal Replacement (ArMORR) study which is a nationally representative prospective cohort study of 10,044 patients who initiated chronic hemodialysis at any of 1,056 U.S. dialysis centers operated by Fresenius Medical Care, North America (“FMC”, Lexington, Mass.) between Jul. 1, 2004 and Jun. 30, 2005. All patients underwent one year of prospective follow up unless they died (15%), underwent kidney transplantation (3%), spontaneously recovered renal function (4%) or transferred to a non-FMC unit prior to completing their first year on hemodialysis (12%).

Data were collected prospectively by clinicians at the point of care and entered into a central database that underwent rigorous quality assurance/quality control auditing mandated by FMC. Data included demographics, comorbidities, routine laboratory results performed by a central lab (Spectra East, Rockleigh, N.J.), and outcomes. ArMORR is unique among dialysis cohorts in that discarded blood samples were stored in liquid nitrogen on all subjects at the initiation of outpatient hemodialysis, enabling analyses of biomarkers not used in clinical practice. The study was approved by the Institutional Review Board of the Massachusetts General Hospital, which waived the need for informed consent given that all data were stripped of personal identifiers before being transferred to the investigators. Study population

Mortality was first examined according to baseline serum phosphate levels in the entire ArMORR cohort. A second analysis included FGF-23 and mortality in a nested case-control sample for which “Cases” are defined as those patients who died during the first year on hemodialysis and “Controls” as those patients who survived the first year on hemodialysis. Based on internal pilot data, it was estimated that 50 Cases and 50 Controls would provide 90% power to detect a standardized difference (μ₁-μ₂/σ) of 0.66 in mean FGF-23 levels with a type I error rate of 5%. Since hyperphosphatemia is a risk factor for mortality, the confounding factor of serum phosphate was minimized by using frequency matching to randomly select 50 Cases and 50 Controls within each Quartile of baseline serum phosphate from the overall cohort. This resulted in a final sample of 200 Cases and 200 Controls providing 90% power to detect an odds ratio of mortality of 1.8 by comparing upper versus lower FGF-23 Quartiles with a type I error rate of=0.05.

FGF-23 secretion may be increased by dietary phosphorus intake and activated vitamin D. Activated vitamin D is used to treat secondary hyperparathyroidism in hemodialysis patients. Kalantar-Zadeh et al., “Survival predictability of time-varying indicators of bone disease in maintenance hemodialysis patients. Kidney Int 70:771-780 (2006); Melamed et al., “Changes in serum calcium, phosphate, and PTH and the risk of death in incident dialysis patients: a longitudinal study” Kidney Int 70:351-357 (2006); Teng et al., “Survival of patients undergoing hemodialysis with paricalcitol or calcitriol therapy” N Engl J Med 349:446-456 (2003); Teng et al., “Activated injectable vitamin D and hemodialysis survival: a historical cohort study” J Am Soc Nephrol 16:1115-1125 (2005); and Tentori et al., “Mortality risk among hemodialysis patients receiving different vitamin D analogs” Kidney Int 70:1858-1865 (2006).

Thus, increased FGF-23 levels in an unselected dialysis patient could reflect high phosphorus exposure (i.e., a factor that increases mortality risk), previous therapy with activated vitamin D (i.e., a factor that decreases mortality risk), or both. Consequently, patients were excluded that had initiated therapy with oral or intravenous activated vitamin D prior to the collection of their baseline blood sample for FGF-23 measurement. Subjects remained eligible if they received activated vitamin D after the baseline blood sample, whereupon phosphorus and vitamin D were analyzed as a covariate. Also excluded were subjects who underwent kidney transplantation and those who spontaneously recovered renal function or were transferred out of the FMC system.

Blacks, Hispanics, and Caucasians were included in the study design. It is believed that

Blacks and Hispanics are at greater risk of developing end stage renal disease compared to non-Hispanic Whites, but upon reaching dialysis, demonstrate a significant survival advantage. Cowie et al., “Disparities in incidence of diabetic end-stage renal disease according to race and type of diabetes” N Engl J Med 321:1074-1079 (1989); Robinson et al., “Revisiting survival differences by race and ethnicity among hemodialysis patients: the Dialysis Outcomes and Practice Patterns Study” J Am Soc Nephrol 17:2910-8 (2006); Frankenfield et al., “Survival advantage for adult Hispanic hemodialysis patients? Findings from the end-stage renal disease clinical performance measures project” J Am Soc Nephrol 14:180-186 (2003). Although there are known differences in PTH and vitamin D metabolism according to race/ethnicity, there is no data on FGF-23 levels and whether these levels differentially impact survival. In order to focus on Black, Hispanic and Caucasian patients, Other races were excluded (n=693). In the final study group (i.e., Case+Control patients), there was 1 Black-Hispanic patient who was analyzed in the Black group (n=119) while the White-Hispanic patients (hereafter called Hispanic, n=42) were examined separately from the remaining non-Hispanic-Whites (hereafter called Caucasian, n=239).

### Exposures, Outcomes and Covariates

The primary exposure was plasma levels of FGF-23 measured in specimens that were collected at the initiation of outpatient hemodialysis and prior to administration of any exogenous activated vitamin D. The primary outcome was all-causes of mortality during the first year of dialysis, confirmed by mandatory discharge diagnosis reports from dialysis centers.

FGF-23 levels were measured in duplicate after a single freeze-thaw cycle in blinded batched assays. This is the first assessment of FGF-23 protein levels and chronic kidney disease mortality risk, although C-terminal FGF-23 (cFGF-23) fragments have been reported to accumulate in kidney disease. Larsson et al., “Circulating concentration of FGF-23 increases as renal function declines in patients with chronic kidney disease, but does not change in response to variation in phosphate intake in healthy volunteers” Kidney Int 64:2272-2279 (2003). Even so, few studies directly compared the results from C-terminal (cFGF-23) and intact FGF-23 (iFGF-23) assays in renal failure.

Both cFGF-23 and iFGF-23 were measured in this study (Immutopics, San Clemente, Calif.; inter-, intra assay coefficients of variation <5%). The cFGF-23 assay detects intact FGF-23 and its C-terminal fragments, while the iFGF-23 assay is specific for the intact molecule. Serum was available for measurement of 1,25D levels by radioimmunoassay in 52 cases and 69 controls (Diasorin, Stillwater, Minn.). Serum phosphate was measured using standard assays and parathyroid hormone (PTH) was measured using Nichols Bio-intact assay that detects full length PTH (1-84, hemodialysis target range 75-150 pg/ml30).

The following case-mix variables were analyzed as covariates: age, sex, race, ethnicity, etiology of renal failure, blood pressure, body mass index, dialysis access at initiation, dialysis dose assessed by the urea reduction ratio, facility specific standardized mortality rates (SMR)21, and comorbidities at the initiation of dialysis (diabetes, hypertension, coronary artery disease, congestive heart failure, chronic obstructive pulmonary disease, non-cutaneous malignancy, stroke). Comorbidities were ascertained by the individual patients' practitioners and derived from the initial intake history, physical examination, and medical records reviewed by the dialysis centers. The following baseline laboratory results were also analyzed as covariates: sodium, potassium, bicarbonate, creatinine, calcium, phosphate, PTH, alkaline phosphatase, albumin, hemoglobin and ferritin. Phosphorus binder use prior to measurement of FGF-23 and subsequent active vitamin D therapy were both analyzed as covariates.

### Statistical Analysis

Descriptive statistics were used to compare baseline demographics and laboratory results in the overall ArMORR cohort, among cases and controls, and according to race/ethnicity. Spearman correlation and linear regression tested the association between cFGF-23 and iFGF-23, using log-transformed values to analyze FGF-23 as a continuous variable. To test for non-linear associations between FGF-23 and mortality and for the purpose of interpretability FGF-23 (in Quartiles) was examined according to its distribution in the overall sample. cFGF-23 and iFGF-23 data were examined in parallel.

A Cox proportional-hazards analysis was used to examine the risk of mortality associated with baseline phosphate levels in the full ArMORR cohort, censoring at the time of kidney transplantation, transfer to a non-FMC center, or recovery of kidney function. A logistic regression was then used to test the association between baseline FGF-23 levels and mortality in the Case-Control sample. Multivariable regression models were used to adjust for potential confounding. The data were first adjusted for case-mix variables and then adjusted for the laboratory test results. Laboratory tests were analyzed on a continuous scale except for PTH, which required log-transformation, and 1,25D levels, which were analyzed in tertiles with an extra category for missing values. Treatment with dietary phosphorus binders can reduce FGF-23 levels. Nagano et al., “Effect of manipulating serum phosphorus with phosphate binder on circulating PTH and FGF23 in renal failure rats” Kidney Int 69:531-537 (2006); and Koiwa et al., “Sevelamer hydrochloride and calcium bicarbonate reduce serum fibroblast growth factor 23 levels in dialysis patients” Ther Apher Dial 9:336-339 (2005). Consequently, the data was further adjusted for phosphorus binder use that preceded the FGF-23 and serum phosphate measurements, and examined models that excluded patients who had been previously treated with binders. Similarly, the data was also adjusted for subsequent treatment with activated vitamin D.

Significant interactions (P<0.05) between FGF-23, race (Black vs. White), ethnicity (Hispanic vs. White) and mortality were further analyzed using logistic regression models stratified by race and models with interaction terms (FGF-23 above or below the population median x race). Analyses were performed using Intercooled Stata 7.0 (Stata Corporation, College Station, Tex.). Two-sided P values <0.05 were considered statistically significant.

### Results

1. Serum Phosphate Levels and Mortality in ArMORR

Baseline characteristics of the 10,044 ArMORR participants at the initiation of hemodialysis are presented in Table 1.

TABLE 1
Characteristics of the ArMORR cohort (n = 10,044) at the initiation
of hemodialysis. Results are reported as mean ± standard deviation,
median (interQuartile range), or proportions, as appropriate.

| | |
|---|---|
| Age | 63 ± 16 |
| Female (%) | 45 |
| Black (%) | 32 |
| Hispanic (%) | 13 |
| Body mass index (kg/m2) | 28 ± 9 |
| Blood pressure (mm Hg) | |
| Systolic | 148 ± 21 |
| Diastolic | 74 ± 14 |
| Etiology of renal failure (%) | |
| Diabetes | 43 |
| Hypertension | 35 |
| Glomerulonephritis | 10 |
| Other | 12 |
| Initial vascular access (%) | |
| Fistula | 26 |
| Graft | 11 |
| Catheter | 63 |
| Co-morbidities (%) | |
| Coronary artery disease | 10 |
| Congestive heart failure | 12 |
| Chronic obstructive pulmonary disease | 2 |
| Malignancy | 3 |
| Stroke | 3 |
| Laboratory tests | |
| Albumin (g/dl) | 3.5 ± 0.5 |
| Calcium (mg/dl) | 8.9 ± 0.8 |
| Phosphate (mg/dl) | 4.7 ± 1.6 |
| Bio-intact PTH (pg/ml) | 206 (111 |
| Alkaline phosphatase (U/L) | 83 (64 |
| Creatinine (mg/dl) | 6.3 ± 2.7 |
| Hemoglobin (g/dl) | 10.3 ± 1.4 |
| Ferritin (ng/ml) | 202 (98 |
| Urea reduction ratio (%) | 68 ± 11 |

The mean serum phosphate level of 4.6±1.6 mg/dl was lower than previous dialysis studies; otherwise, baseline characteristics were comparable to previous reports.

Seventeen percent (17%) of patients had been initiated on dietary phosphorus binders and 3% were on activated vitamin D prior to the collection of their baseline blood sample for ArMORR. The overall 1-year mortality rate was 178 deaths/1000 patient-years at risk. Serum phosphate levels were significantly lower at baseline in patients who died during the subsequent year on dialysis compared with those who survived (4.4±1.6 vs. 4.7±1.6 mg/dl; P<0.01). Although serum phosphate levels in the lowest Quartile (<3.5 mg/dl) were associated with increased risk of mortality compared to levels of 3.5-4.5 mg/dl in a univariate analysis (HR 1.4, 95% CI 1.2, 1.6), the risk was attenuated when adjusted for case-mix variables and laboratory results (HR 1.1; 95% CI 0.9, 1.2). In contrast, serum phosphate levels in the highest Quartile (>5.5 mg/dl) were associated with increased risk of mortality compared to normal levels after similar multivariable adjustment (HR 1.2; 95% CI 1.1, 1.4).

2. Characteristics of the Case-Control Sample

Baseline characteristics of the nested Case-Control sample are presented in Table 2.

TABLE 2
Characteristics of the nested Case-Control sample at the initiation
of hemodialysis. Results are reported as mean ± standard deviation,
median (interQuartile range), or proportions, as appropriate.

| | Died | Survived | |
|---|---|---|---|
| Characteristic | N = 200 | N = 200 | P |
| Age | 70 ± 14 | 61 ± 15 | <0.01 |
| Female (%) | 47 | 46 | NS |
| Black (%) | 24 | 36 | 0.01 |
| Hispanic (%) | 7 | 15 | 0.02 |
| Body mass index (kg/m²) | 28 ± 6 | 27 ± 11 | 0.02 |
| Blood pressure (mm Hg) | | | |
| Systolic | 140 ± 27 | 146 ± 21 | <0.01 |
| Diastolic | 70 ± 16 | 76 ± 13 | <0.01 |
| Etiology of renal failure (%) | | | |
| Diabetes | 42 | 44 | NS |
| Hypertension | 40 | 37 | NS |
| Glomerulonephritis | 6 | 11 | NS |
| Other | 14 | 9 | NS |
| Initial vascular access (%) | | | |
| Fistula | 14 | 36 | <0.01 |
| Graft | 10 | 11 | NS |
| Catheter | 76 | 53 | <0.01 |
| Co-morbidities (%) | | | |
| Coronary artery disease | 14 | 8 | NS |
| Congestive heart failure | 21 | 10 | <0.01 |
| Chronic obstructive | 3 | 2 | NS |
| pulmonary disease | | | |
| Malignancy | 6 | 3 | NS |
| Stroke | 6 | 4 | NS |
| Laboratory tests | | | |
| Albumin (g/dl) | 3.2 ± 0.5 | 3.4 ± 0.6 | <0.01 |
| Calcium (mg/dl) | 8.4 ± 0.8 | 8.3 ± 0.9 | NS |
| Phosphate (mg/dl) | 4.4 ± 1.7 | 4.4 ± 1.6 | NS |
| Bio-intact PTH (pg/ml) | 192 (101, 326) | 198 (113, 343 | NS |
| Alkaline phosphatase (U/L) | 86 (68, 126) | 89 (68, 112) | NS |
| Creatinine (mg/dl) | 5.4 ± 2.3 | 6.3 ± 2.6 | <0.01 |
| Hemoglobin (g/dl) | 10.1 ± 1.3 | 10.2 ± 1.3 | NS |
| Ferritin (ng/ml) | 218 (94, 480) | 199 (88, 332) | NS |
| Urea reduction ratio (%) | 69 ± 11 | 69 ± 11 | NS |

Compared to survivors, patients who died were older, had lower blood pressure and body mass index, and lower levels of albumin and creatinine; a higher proportion were Caucasian and had a history of congestive heart failure and a catheter for dialysis access. There were no significant differences in baseline characteristics comparing the cases and controls that were randomly selected for the current study with the overall population of eligible cases and controls (data not shown).

3. Fibroblast Growth Factor-23 Levels and Mortality

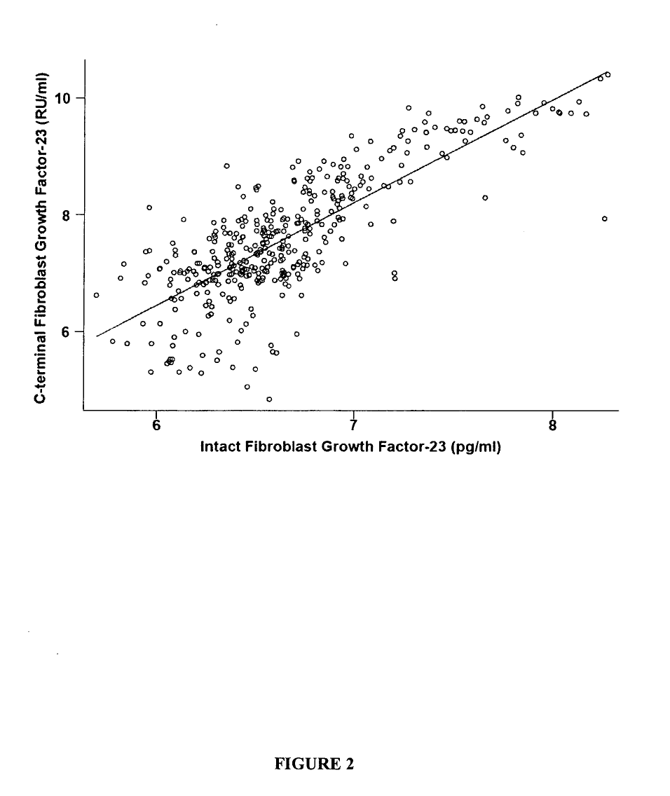

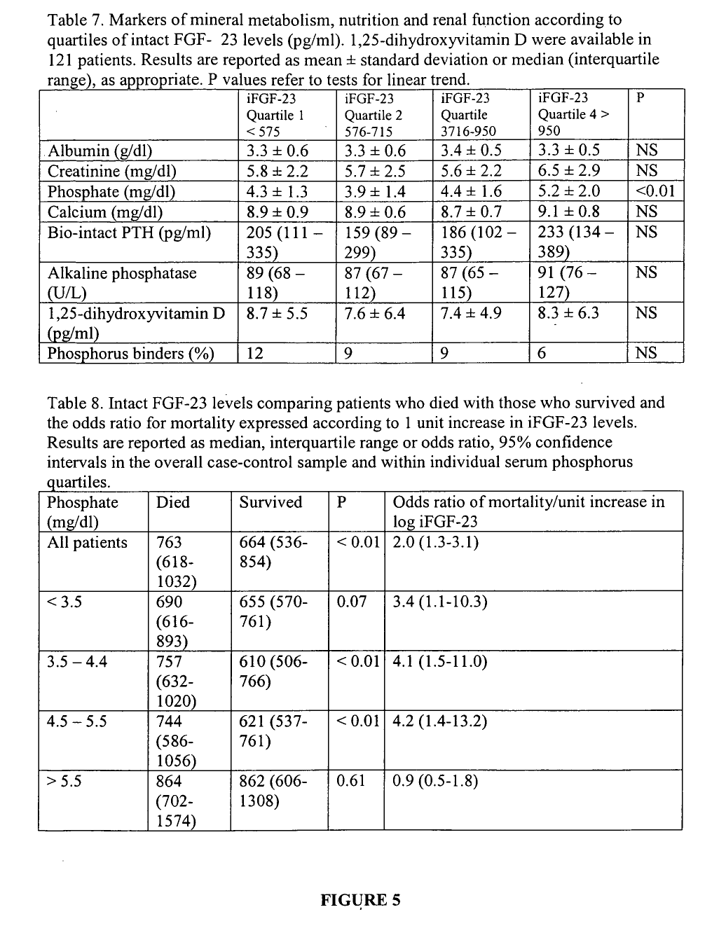

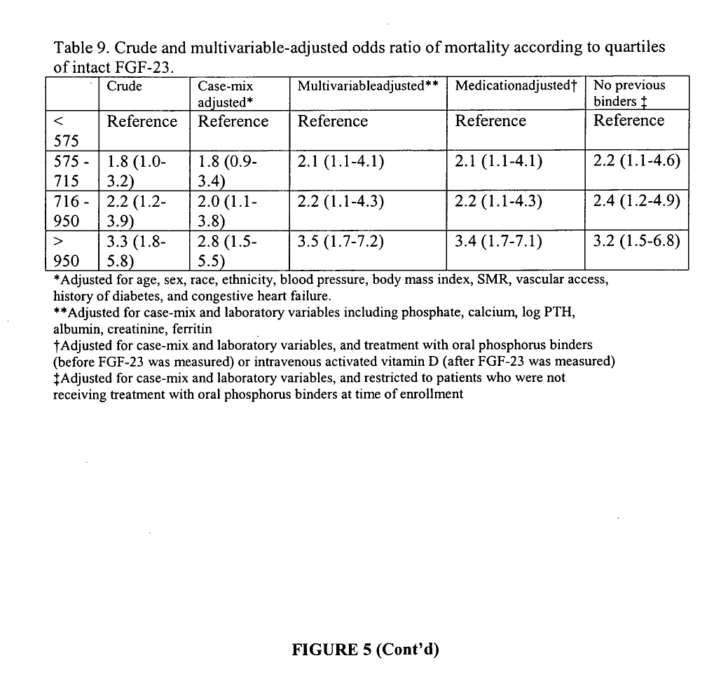

There was strong linear correlation (r=0.74, P<0.01) between cFGF-23 across all FGF-23 Quartiles (median 1752; interQuartile range [IQR]=1089-4019 RU/ml) and iFGF-23 (713; IQR=579-951 pg/ml) levels. See, FIG. 2. The results of all subsequent analyses were qualitatively similar when measuring either C-terminal FGF-23 or intact FGF-23. Therefore, the primary focus was on FGF-23 C-terminal results. The iFGF-23 raw data are presented in Supplemental Table 7. See, FIG. 5.

Markers of mineral metabolism, nutrition and renal function according to Quartiles of cFGF-23 are presented in Table 3.

TABLE 3
Markers of mineral metabolism, nutrition and renal function according to Quartiles
of c-terminal FGF-23 levels (RU/ml). 1,25-dihydroxyvitamin D levels were available
in 121 patients. Results are reported as mean ± standard deviation or median
(interQuartile range), as appropriate. P values refer to tests for linear trend.

| | cFGF-23 | cFGF-23 | cFGF-23 | cFGF-23 | |
|---|---|---|---|---|---|
| | Quartile 1 | Quartile 2 | Quartile 3 | Quartile 4 | |
| | <1090 | 1090-1750 | 1751-4010 | >4010 | P |
| Albumin (g/dl) | 3.4 ± 0.5 | 3.3 ± 0.5 | 3.3 ± 0.6 | 3.3 ± 0.5 | NS |
| Creatinine (mg/dl) | 5.3 ± 2.2 | 5.4 ± 2.2 | 6.3 ± 2.2 | 6.6 ± 2.9 | <0.01 |
| Phosphate (mg/dl) | 3.9 ± 1.2 | 4.1 ± 1.3 | 4.5 ± 1.6 | 5.2 ± 2.1 | <0.01 |
| Calcium (mg/dl) | 8.8 ± 0.8 | 8.9 ± 0.7 | 8.8 ± 0.8 | 9 ± 0.8 | NS |
| Bio-intact PTH (pg/ml) | 180 (99-313) | 137 (95-273) | 253 (119-377) | 234 (145-435) | NS |
| Alkaline phosphatase (U/L) | 82 (64-103) | 89 (67-114) | 91 (69-114) | 100 (77-131). | <0.01 |
| 1,25-dihydroxyvitamin D | 9.1 ± 5.2 | 6.9 ± 6 | 8.4 ± 5 | 7.5 ± 5.8 | NS |
| (pg/ml) | | | | | |
| Phosphorus binders (%) | 12 | 8 | 9 | 6 | NS |

There was a linear increase in serum phosphate, creatinine and alkaline phosphatase levels with increasing Quartiles of cFGF-23 but no significant linear trends between FGF-23 and calcium, PTH, 1,25D, or albumin. Median cFGF-3 levels were significantly higher in patients who died versus those who survived in the overall sample and within each individual serum phosphate Quartile except the highest (Table 4). The iFGF-23 raw data are presented in Supplemental Table 8. See, FIG. 5.

TABLE 4
C-terminal FGF-23 levels comparing patients who died with those who survived
and the odds ratio for mortality expressed according to 1 unit increase
in cFGF-23 levels. Results are reported as median, interQuartile range
or odds ratio, 95% confidence intervals in the overall case-control sample
and within individual serum phosphate Quartiles.

| | | | | Odds ratio of mortality/ |
|---|---|---|---|---|
| Phosphate | | | | unit increase |
| (mg/dl) | Died | Survived | P | in log cFGF-23 |
| All patients | 2260 (1196-5296) | 1406 (989-2741) | <0.01 | 1.5 (1.2-1.8) |
| <3.5 | 1790 (1175-3941) | 1148 (927-2169) | 0.01 | 1.8 (1.2-2.8) |
| 3.5-4.4 | 2049 (1109-4865) | 1131 (893-1629) | <0.01 | 1.8 (1.2-2.7) |
| 4.5-5.5 | 2207 (1186-5238) | 1499 (1044-2262) | 0.02 | 1.8 (1.1-3.0) |
| >5.5 | 3541 (1871-10491) | 2686 (1527-6210) | 0.29 | 1.1 (0.7 1.6) |

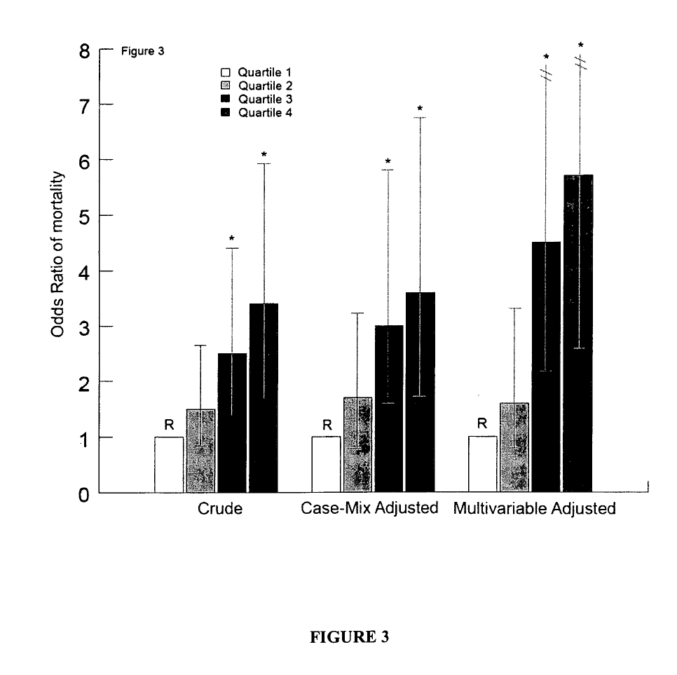

In univariate analyses, increased cFGF-23 levels were associated with increased risk of mortality in the overall population (OR/unit increase in log cFGF-23: 1.5; 95% CI 1.2, 1.8) and consistently across the individual serum phosphate Quartiles except the highest (Table 4). In the overall sample, FGF-23 remained significantly associated with mortality when adjusted for case mix variables (OR/unit increase in log cFGF-23: 1.6; 95% CI 1.2, 1.9), and when further adjusted for baseline laboratory results (OR/unit increase in log cFGF-23 1.8; 95% CI 1.4, 2.4). When FGF-23 was examined in Quartiles, there was a monotonic increase in risk of mortality with increasing cFGF-23 in univariate, case-mix adjusted and fully adjusted models (FIG. 3, Table 5). The iFGF-23 raw data are presented in Supplemental Table 9. See, FIG. 5.

TABLE 5
Crude and multivariable-adjusted odds ratio of mortality
according to Quartiles of C-terminal FGF-23.

| | | Case-mix | Multivariable | Medication | No previous |
|---|---|---|---|---|---|
| | Crude | adjusted* | adjusted** | adjusted† | binders‡ |
| <1090 | Reference | Reference | Reference | Reference | Reference |
| 1090-1750 | 1.5 (0.9-2.7) | 1.7 (0.8-3.2) | 1.6 (0.8-3.3) | 1.6 (0.8-3.1) | 1.4 (0.9-2.8) |
| 1751-4010 | 2.5 (1.4-4.4) | 3.0 (1.6-5.8) | 4.5 (2.2-9.4) | 4.8 (2.3-10.1) | 4.2 (1.9-9.0) |
| >4010 | 3.4 (1.9-5.9) | 3.6 (1.9-6.9) | 5.7 (2.6-12.6) | 5.5 (2.5-12.2) | 5.1 (2.3-11.6) |

*Adjusted for age, sex, race, ethnicity, blood pressure, body mass index, SMR, vascular access, history of diabetes, and congestive heart failure.
**Adjusted for case-mix and laboratory variables including phosphate, calcium, log PTH, albumin, creatinine, ferritin
†Adjusted for case-mix and laboratory variables, and treatment with oral phosphorus binders (before FGF-23 was measured) or intravenous activated vitamin D (after FGF-23 was measured)
‡Adjusted for case-mix and laboratory variables, and restricted to patients who were not receiving treatment with oral phosphorus binders at time of enrollment

In addition, the results were qualitatively unchanged when further adjusted for subsequent therapy with activated vitamin D, prior therapy with phosphorus binders, and when subjects who had previously been treated with binders were excluded (Table 5).

4. Effect Modification by Race/Ethnicity

Black and Hispanic patients demonstrated a significant survival advantage compared to Caucasian in ArMORR and in the case-control sample (Black: OR 0.5, 95% CI 0.3, 0.8; Hispanic: OR 0.4, 95% CI 0.2, 0.7). While there were no significant differences in baseline calcium or phosphate levels according to race/ethnicity, Blacks had significantly decreased FGF-23 and increased PTH levels compared to Caucasians, and were most likely to be on phosphorus binders at the initiation of dialysis (Table 6).

TABLE 6
Baseline mineral metabolism variables at the initiation of dialysis according to race/ethnicity.
Results are reported as mean ± standard deviation or median (interQuartile range),
as appropriate. refers to significant differences compared with White.

| | Caucasian N = 239 | Black N= 119 | Hispanic N = 42 |
|---|---|---|---|
| Calcium (mg/dl) | 8.9 ± 0.7 | 8.8 ± 0.9 | 8.9 ± 0.9 |
| Phosphate (mg/dl) | 4.5 ± 1.7 | 4.2 ± 1.4 | 4.8 ± 2.1 |
| PTH (pg/ml) | 172 (99-279) | 321 (147-465)* | 158 (111-258) |
| Alkaline phosphatase (U/L) | 87 (67-114) | 89 (68-125) | 95 (79-124) |
| cFGF-23 (RU/ml) | 2016 (1132-4865) | 1579 (966-2959)* | 1336 (1094-2262)* |
| 1,25-dihydroxyvitamin D (pg/ml) | 7.7 ± 5.4 | 8.5 ± 6.1 | 6.7 ± 5.3 |
| Phosphorus binders (%) | 6 | 13* | 14 |

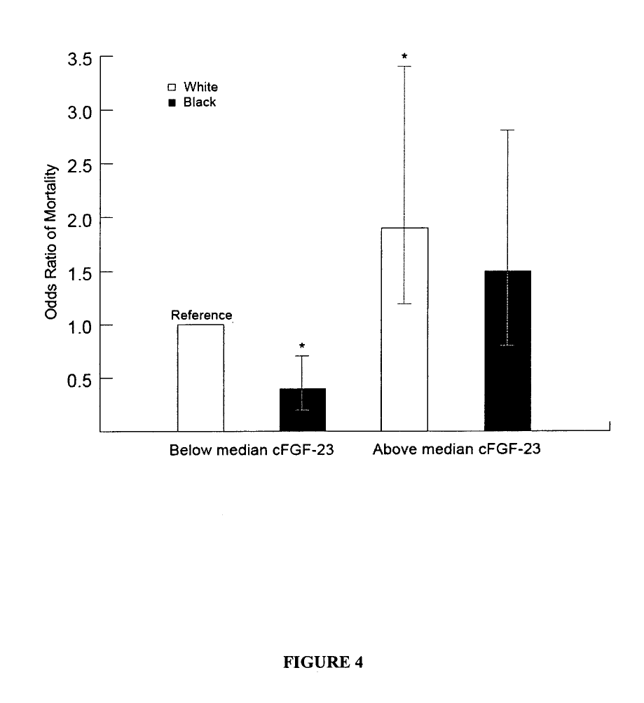

Although there was no interaction between ethnicity, FGF-23 and mortality, there was significant interaction between race and cFGF-23 (P<0.05). Among patients with FGF-23 levels below the population median, Blacks demonstrated a 60% lower risk of mortality compared to Caucasians, whereas the risk of death according to race was similar in subjects with high FGF-23 levels (FIG. 4).

### EXAMPLE II

### Effects of Dietary Phosphorus Intake on FGF-23 Levels

In this example, data is presented showing that dietary phosphorus intake correlates with FGF-23 in pre-dialysis CKD and FGF-23 levels increase as glomerular filtration rate (GFR) declines. This example explores the hypothesis that may account for the observation that FGF-23 and PTH levels increase long before hyperphosphatemia or hypocalcemia first appear in CKD (60, 68).

CKD patients who ingest a high phosphorus diet relative to their nephron mass may recruit a vigorous FGF-23 secretory response that induces greater per-nephron phosphate excretion to maintain normophosphatemia; the compensatory increase in FGF-23 levels inhibits renal 1,25D synthesis and thereby initiates sHPT in the absence of hyperphosphatemia.

A randomized, controlled trial will be performed to determine if decreasing phosphorus intake in normophosphatemic stage 3-4 CKD patients using phosphate binders, dietary restriction, or a combination of both will decrease FGF-23 levels, lead to increased 1,25D production and thus decreased PTH.

### EXAMPLE III

### FGF-23 Precedes Hyperphosphatemia in Chronic Kidney Disease Patients

In this example, data is presented showing that increased FGF-23 precedes hyperphosphatemia and secondary hyperparathyroidism (sHPT) in CKD and is associated with increased parathyroid hormone (PTH) levels independent of GFR.

C-terminal FGF-23, intact PTH, 25D, 1,25D, phosphate and fractional excretion of phosphate (FEpo4) was measured in a cross-sectional study of 80 pre-dialysis patients from across the spectrum of CKD.

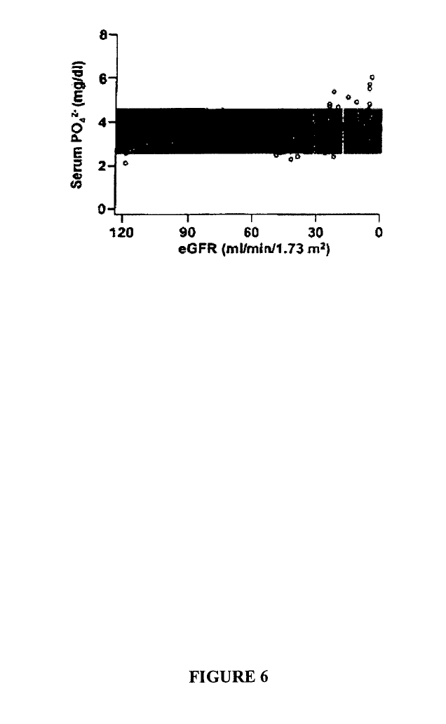

Hyperphosphatemia was present in only 12% of subjects, all of whose eGFR was <30 ml/min See, FIG. 6. Even among subjects with GFR <30, most had a normal serum phosphate (shaded). FGF-23 was inversely associated with eGFR (P<0.01) and levels were increased above the normal range (˜50 RU/ml) at all GFR levels (>60 ml/min: 86±61; 45-60:136±69; 30-44:224±200; <30: 436±494 RU/ml). FEpo4 correlated inversely with GFR (P<0.01), and FGF-23 was the primary factor (P<0.01) associated with FEpo4, suggesting that increased FGF-23 is the primary factor that augments FEpo4 in CKD. 1,25D levels decreased as GFR declined and FGF-23 was the strongest independent predictor of 1,25D. Indeed, adjusting for FGF-23 completely extinguished the association between GFR and 1,25D, suggesting that FGF-23 is a central mechanism of 1,25D deficiency that begins in early CKD (60).

FGF-23 levels increase early in CKD long before hyperphosphatemia develops. Increased FGF-23 helps maintain normal serum phosphate levels in CKD by inducing phosphaturia and inhibiting 1,25D synthesis, but the latter leads to sHPT. Thus, excessive phosphorus intake, leading to excessive FGF-23 secretion, may play a role in the early pathogenesis of sHPT in CKD.

### EXAMPLE IV

### FGF-23 as a Prognostic Indicator in Pre-Dialysis Chronic Kidney Disease Patients

In this example, data is presented showing that increased FGF-23 levels are independently associated with kidney disease progression, left ventricular hypertrophy, coronary artery calcification and mortality in pre-dialysis CKD patients.

### EXAMPLE V

### FGF-23 is a Superior Biomarker as Compared to Phosphorous

In this example, data between phosphorus Quartiles and FGF-23 Quartiles are compare showing that the associations with cardiovascular and renal end-points are stronger for FGF-23 compared with contemporaneous serum phosphate measurements. Consequently, FGF-23 is a superior biomarker as compared to phosphorous levels.

A comparison the phosphate Quartile data to the FGF-23 Quartile data presented in Example I, shows that FGF-23 has a multiple adjusted variable mortality risk of 600%, whereas comparable data using phosphate levels as an indication shows only a 20% risk. This data demonstrates that FGF-23 has a 30-fold improved sensitivity to predict CKD mortality as compared to phosphate levels.

### EXAMPLE VI

### FGF-23 and Left Ventricular Hypertrophy in CKD

This example provides data showing the FGF-23 levels are associated with LVH in chronic kidney disease patients.

Coronary artery calcification (CAC) was measured by multiplane cardiac CT and LVH by echocardiography in a cross-sectional study of 162 pre-dialysis CKD patients (age >30 years, eGFR <60 ml/min) free of known coronary artery disease or angina. CAC scores were examined as a continuous score and as mild/moderate (<100) or severe (≧100) (121). LVH was defined as a left ventricular mass index (LVMI) ≧134 g/m² in men and ≧110 g/m² in women (122). C-terminal FGF-23 (cFGF-23), phosphate, creatinine, calcium, albumin, cholesterol, and intact PTH were also determined. Characteristics of the study population are presented in the Table 7.

TABLE 7
Study population characteristics

| | N = 162 |
|---|---|
| Variable | |
| Age | 63 ± 11 |
| Female (%) | 32 |
| Black (%) | 39 |
| eGFR (ml/min/1.73 m²) | 34 ± 11 |
| BMI (kg/m 2) | 31 ± 7 |
| SBP (mmHg) | 135 ± 20 |
| Laboratory tests | |
| Creatinine (mg/dl) | 2.2 ± 0.9 |
| Phosphate (mg/dl) | 3.7 ± 0.7 |
| Calcium (mg/dl) | 9.1 ± 0.8 |
| Albumin (g/dl) | 3.8 ± 0.7 |
| Cholesterol (g/dl) | 177 ± 44 |
| iPTH (pg/ml) | 70 (47-09) |
| cFGF-23 (RU/ml) | 119 (80-222) |

The median cFGF-23 of 119 RU/ml was more than double the normal range (54). 52% of patients had severe CAC, of which the strongest correlate was increased age. The highest vs. the lowest FGF-23 tertile was associated with a significantly increased risk of severe CAC (OR 2.4, 95% CI 1.1-5.5). Although the significance was attenuated when adjusted for age (OR 2.4, CI 0.9-5.9), the point estimate was unchanged suggesting a need for further studies with greater power.

Mean LVMI was 90+24 g/m² in men and 81+21 g/m² in women; 4% of men and 10% of women had LVH. In univariate analyses, log FGF-23 was associated with increased LVMI (6% increase/unit increase in log FGF-23, P=0.01). When adjusted for age, gender, race, eGFR, diabetes, BP and BMI, log FGF-23 remained strongly associated with increased LVMI (8% increase/unit increase in log FGF-23 levels, P<0.01). When further adjusted for cholesterol, phosphate, PTH, and NT-proBNP levels, log FGF-23 was the only independent predictor of increased LVMI (8% increase/unit increase in log FGF-23, P<0.01). In addition, log FGF-23 was associated with an increased risk of LVH in men (OR 1.8, 95% Cl 0.7, 4.5) and women (OR 4.6, 95% Cl 1.2, 1.8), although the OR's did not reach significance in men, likely because of limited power.

Increased FGF-23 was linearly associated with increased LVMI independent of known risk factors including age, BP, hemoglobin, and eGFR. While further studies are required in larger populations, there was also a trend towards an association between increased FGF-23 and LVH and severe CAC. These data suggest that FGF-23 may be a marker of CVD.

### EXAMPLE VII

### FGF-23 in Post-Transplant Hypophosphatemia

Hypophosphatemia due to urinary phosphate wasting occurs in up to 93% of kidney transplant recipients during the first few months (85, 86). Tertiary hyperparathyroidism has been thought to be the etiology but hypophosphatemia occurs despite low PTH and can persist after high PTH normalizes (86-89). Furthermore, 1,25D levels remain low post-transplant despite excessive PTH and hypophosphatemia, each of which should stimulate 1,25D production by the healthy allograft (90, 91). This example provides data showing whether increased FGF-23 levels account for hypophosphatemia and decreased 1,25D levels in a post-transplant period.

A prospective, longitudinal study of 27 living donor transplant recipients was followed for six months post-transplant. C-terminal FGF-23, intact PTH, 25D, 1,25D, phosphate and FEpo4 were measured. (84)

Hypophosphatemia <2.5 mg/dl developed in 85% of subjects, including one who had previously undergone parathyroidectomy; 37% developed levels ≦1.5 mg/dl. The mean pre-transplant FGF-23 level of 1,218±542 RU/ml decreased to 557±579 RU/ml within the first week following transplantation, but was still markedly above normal (˜50 RU/ml) (54). FGF-23 was independently associated with serum phosphate (P<0.01), FEpo4 (P<0.01) and decreased 1,25D levels (P<0.01); PTH was not independently associated with any of these parameters. An area under the FGF-23 curve between the pre- and first post-transplant levels that was greater than the median was associated with a relative risk of developing hypophosphatemia of 5.3 (P=0.02) compared with lower levels. Area under the PTH curve was not associated with hypophosphatemia.

The data show that excessive FGF-23 exposure is associated with post-transplant hypophosphatemia, further supporting a role for FGF-23 in phosphate metabolism in CKD.

### EXAMPLE VIII

### Vitamin D Levels and Early Mortality in Hemodialysis Patients

Vitamin D deficiency is believed associated with diabetes, malignancy, and CVD, including hypertension, LVH, congestive heart failure, and excessive activation of the rennin-angiotensin system (103-110). Although CVD is the leading cause of death on dialysis (111) and deficiencies in the vitamin D axis are common in CKD (112), the association between vitamin D levels and dialysis outcomes were unknown. This example testes the hypothesis that decreased levels of 25D (storage form) and 1,25D (active hormonal form) at the initiation of dialysis are associated with increased risk for 90-day all-cause and CVD mortality. (102)

A prospective, nested case-cohort study was performed within ArMORR. 25D (RIA, Diasorin Inc., Stillwater, Minn.; CV <3%) and 1,25D (RIA, American Medical Laboratories, Chantilly, Va.; CVs <6.5%) levels were measured in baseline serum samples of 250 consecutive incident hemodialysis patients who died within 90 days of initiating hemodialysis and a random selection of 750 patients who survived at least 90 days. Subjects were excluded if they had received active vitamin D prior to the collection of the serum sample for vitamin D levels; therapy that began after the levels were measured was examined as a covariate, and effect modification of the association between vitamin D levels and survival by vitamin D therapy was examined. The data was adjusted for age, gender, race, etiology of renal failure, BP, BMI, dialysis access, urea reduction ratio, facility specific mortality rates, co-morbidities at the initiation of dialysis (diabetes, hypertension, CVD, COPD, cancer and liver disease) and the results of standard laboratory tests obtained at the initiation of dialysis. Since Vitamin D levels are influenced by climate, the data was also adjusted for season (summer:. April 1 September 30 versus winter: October 1 March 30) and latitude of the state in which patients initiated dialysis.

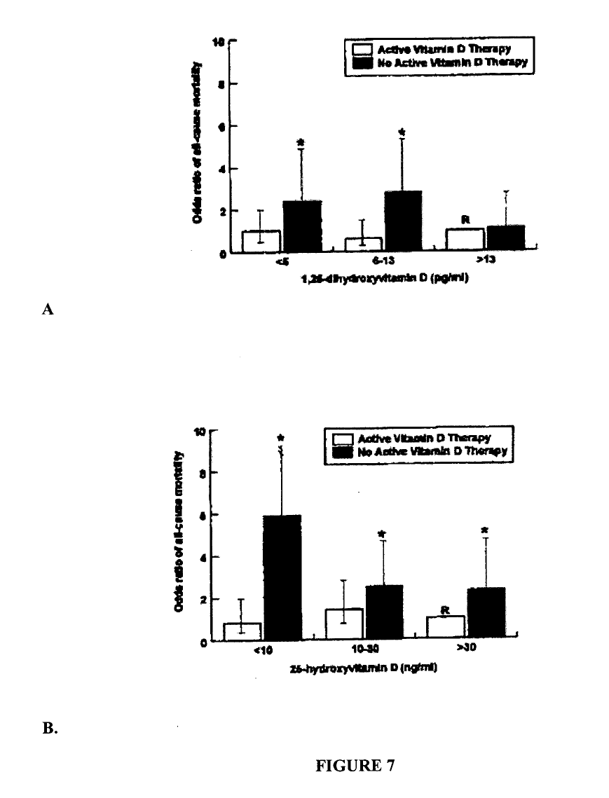

Mean 25D and 1,25D levels were 21±13 ng/ml and 11±10 pg/ml, respectively; 78% of patients were vitamin D deficient (25D<30 ng/ml) and 18% were severely deficient (25D<10 ng/ml). Calcium, phosphate and PTH levels correlated poorly with 25D and 1,25D levels (r<0.20 each). While low vitamin D levels were associated with increased mortality, significant interaction was noted between baseline vitamin D levels, subsequent active vitamin D therapy (median duration 74 days), and survival (P≦0.01 for both 25D and 1,25D). Compared to patients with normal 25D levels (>30 ng/ml) or 1,25D levels in the upper tertile (>13 pg/ml) who received active vitamin D therapy, untreated patients with 25D<10 ng/ml (OR 5.9; CI 2.6, 13.7) or 1,25D<6 pg/ml (OR. 2.1 Cl 1.0, 4.6) were at significantly increased multivariable-adjusted risk for early mortality. The results were exaggerated further when restricted to CVD-related mortality. See, FIG. 7.

Among incident hemodialysis patients, deficiencies in the vitamin D axis were common, weakly correlated with other components of mineral metabolism, and independently associated with increased allcause and CVD-related mortality. Although it is not necessary to understand the mechanism of an invention, it is believed that since FGF-23 inhibits 1,25D production, increased FGF-23 levels may mediate a link between the excess mortality associated with hyperphosphatemia and 1,25D deficiency (37, 63, 102).

### EXAMPLE IX

### FGF-23 Assays

There are currently several types of commercially available ELISA assays to measure circulating FGF-23 levels in humans.

1. Immunotopics San Clemente Calif.

One detects two epitopes in the C-terminus (CV <8%). In addition to intact FGF-23 (iFGF-23), this assay detects C-terminal fragments (cFGF-23), which are catabolic byproducts of intact FGF-23. A second ELISA detects intact FGF-23 (iFGF-23) exclusively because the 2 epitopes flank the metabolic cleavage site of FGF-23 (CV <8%) (54, 55).

2. Kainos Labs

This FGF-23 ELISA Kit is a two-site enzyme-linked immunosorbent assay kit for the measurement of FGF-23 in serum. Yamazaki et al., “Antibody Against Fibroblast Growth Factor-23” United States Patent Application Publication No. 2005/0048058 (herein incorporated by reference). Two specific murine monoclonal antibodies bind to full-length FGF-23. One antibody is immobilized onto the microtiter plate well for capture. The other antibody is conjugated to horseradish peroxidase for detection. In first reaction, a sample containing FGF-23 is incubated with the immobilized antibody in a microtiter well. FGF-23 in the sample is captured with the antibody. At the end of this reaction, the well is washed to remove unbound FGF-23 and other components. In second reaction, this immobilized FGF-23 is incubated with HRP labeled antibody to form a “sandwich”complex:

Anti-FGF-23 antibody:N-terminal FGF-23 C-terminal:HRP labeled anti FGF-23 antibody

At the end of this reaction, the well is washed to remove unbound components. In enzyme reaction, the sandwich complex immobilized on the well is incubated with a substrate solution and then measured by a spectrophotometric microtiter plate reader. The enzymatic activity of the complex bound to the well is directly proportional to the amount of FGF-23 in the sample. A standard curve is generated by plotting the absorbance versus the each concentration of FGF-23 standard. The concentration of FGF-23 in the sample is determined from this curve.

3. Discussion

Since C-terminal fragments of FGF-23 accumulate in CKD (59), measuring cFGF-23 in CKD may less accurately reflect biologically active FGF-23 concentrations than results from iFGF-23 assays. Alternatively, since C-terminal fragments may retain some biological activity (112), cFGF-23 may provide a superior assay target. In the few studies that examined both iFGF-23 and cFGF-23, there was strong linear correlation between assays (up to r=0.97) (159).

The data presented herein shows that the analyses of FGF-23 and mortality were qualitatively identical for cFGF-23 and iFGF-23. Although it is not necessary to understand the mechanism of an invention, it is believed that measurements of iFGF-23 are superior for physiological studies in which subtle “acute” changes in FGF-23 secretion are of interest but could be obscured by accumulated C-terminal fragments.

However, assessing C-terminal fragments that derive from catabolism of intact FGF-23 that was previously secreted in response to phosphorus intake may also provide informative data. For example, cFGF-23 could provide a time-averaged measure of net dietary phosphorus exposure.

### EXAMPLE X

### Physiological Assays

Assays of serum phosphate and other routine metabolic markers using commercially available multi-analyte auto-analyzers (CVs <3%). Spot urinary phosphate excretion is used to calculate FE_{PO4}=(U_{PO4})×(S_{Cr})/(U_{Cr})×(S_{PO4}) a measure of phosphate wasting :standardized the contemporaneous serum phosphate and urine concentration. In patients with normal serum phosphate levels, calculation of the renal threshold for phosphate reabsorption (T_{mPO4}/GFR) may be preferable (160). The T_{mPO4}/GFR is the serum level above which phosphaturia occurs. T_{mPO4}/GFR may be calculated using a standard nomogram (160). Total PTH will be measured using an intact (1-84, 7-84.) assay (Scantibodies, Santee, Calif.).

### EXAMPLE XI

### Phosphate Binders Improve Mortality Risk

This example investigates whether hyperphosphatemia is a risk factor for mortality on dialysis. Although dietary phosphorus binders are FDA-approved and commonly used to treat hyperphosphatemia among dialysis patients, it is unknown if these treatments improve their survival. A hypothesis was tested such that early utilization of dietary phosphorus binders are associated with a subsequent improvement in one-year all-cause mortality among incident hemodialysis patients.

### Methods

A prospective, observational study of phosphorus binder use and mortality was performed utilizing the ArMORR (Accelerated Mortality on Renal Replacement) study. Briefly, ArMORR is a nationally representative prospective cohort study of 10,044 patients who initiated chronic hemodialysis at any of 1,056 US dialysis centers operated by Fresenius Medical Care, North America between Jul. 1, 2004 and Jun. 30, 2005. The survival of 8,610 patients was grouped according to whether they were (n=3555) or were not (n=5055) prescribed phosphorus binders during the first 90 days of outpatient hemodialysis using a pseudo-intention to treat approach. The remaining 1434 patients had initiated phosphorus binders prior to initiating dialysis and were excluded.

The effect of phosphorus binders on mortality in the overall population was assessed using Cox proportional hazards modeling, censoring for kidney transplantation, spontaneous recovery of renal function or transfer to a non-FMC unit prior to completing the first year on hemodialysis. Phosphorus binder use was also examined as a time-dependent exposure to account for those patients who initiated therapy after the first 90 days. Multivariable analyses adjusted for case-mix variables (i.e., for example, age, gender, race, ethnicity, etiology of renal failure, blood pressure, body mass index, vascular access, history of coronary artery disease, or congestive heart failure), baseline laboratory results (i.e., for example, phosphate, calcium, PTH, albumin, creatinine, hemoglobin) and prior and subsequent therapy with activated vitamin D (modeled as a time-dependent covariate).

To further address potential selection bias, a logistic regression was used to calculate a propensity score of the likelihood of receiving phosphorus binders during the first 90 days on dialysis based on patients characteristics and laboratory results upon initiating dialysis. A subcohort of binder users (n=3458) and non-users (n=3458) was generated by one-to-one matching patients by their propensity score±0.03. Kaplan-Meier analyses were used to compare survival in the subcohort, which was well matched on virtually all baseline characteristics. Given residual differences in mean phosphate levels in the matched cohort, stratified analyses by serum phosphate were performed. Finally, to assess whether the effect of binders on survival was mediated by changes in serum phosphate levels, a change in point estimate was analyzed for binder therapy after adjusting for time-varying serum phosphate levels. As a sensitivity analysis, the main analyses was repeated after excluding patients who died during the first 90 days of initiation of hemodialysis.

ArMORR Baseline Statistics

Overall, in comparision to untreated controls, phosphorus binder users: i) were younger (60 vs. 64 years, p<0.001); ii) had higher systolic BP (147 vs. 144 mm Hg, p<0.001); iii) had higher rates of AV fistula for access (27 vs. 23%, p<0.001); iv) had lower rates of coronary artery disease (9 vs. 11%, p=0.001); v) had lower rates of congestive heart failure (10 vs. 13%, p<0.0001); vi) had higher serum phosphate levels (4.9 vs. 4.4 mg/dl; p<0.001); vi) had higher serum bio-intact PTH levels (223 vs. 195 pg/ml; p=0.009); and vii) had higher serum creatinine levels (6.8 vs. 5.9 mg/dl; p<0.001).

A crude hazard ratio (HR) was calculated for one-year mortality comparing binder users vs. non users as 0.58 (95% CI 0.52-0.66; p<0.0001). Binder use remained independently associated with decreased one-year mortality in the multivariable analysis showing an HR of 0.70; 95% CI 0.62-0.79; p<0.0001). Adjusting for any subsequent therapy with activated vitamin D did not alter the results. Binder use also remained independently associated with decreased one-year mortality in fully adjusted models when accounting for its initiation after 90 days on dialysis by modeling it as a time dependent factor showing an HR of 0.82 (95% CI 0.73-0.93; p=0.002). Exclusion of patients with an early mortality (prior to 90 days on dialysis) showed an HR of 0.80 (95% CI 0.68-0.95; p=0.01).

In the propensity score-matched subcohort, virtually all differences in baseline characteristics between binder users and non-users were eliminated. Exceptions included minor, clinically irrelevant differences in age (61 vs. 61 years; p=0.03), creatinine (6.8 vs. 6.6 mg/dl; p=0.02) and baseline serum phosphate (4.9 vs. 4.8 mg/dl; p<0.01).

Serum Phosphate & Mortality From Selected Subsets

The above overall data provide that the ArMORR study contained a well balanced cohort. Consequently, subsets of patients were then selected on the basis of plasma phosphate level and analyzed using unadjusted Kaplan-Meier curves with log rank tests to compare survival rates between phosphate binder users and non-users.

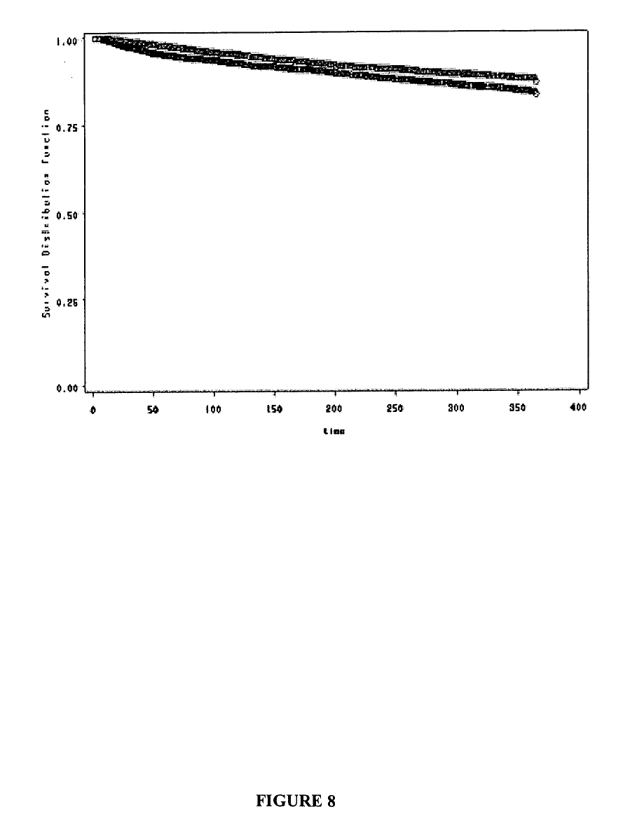

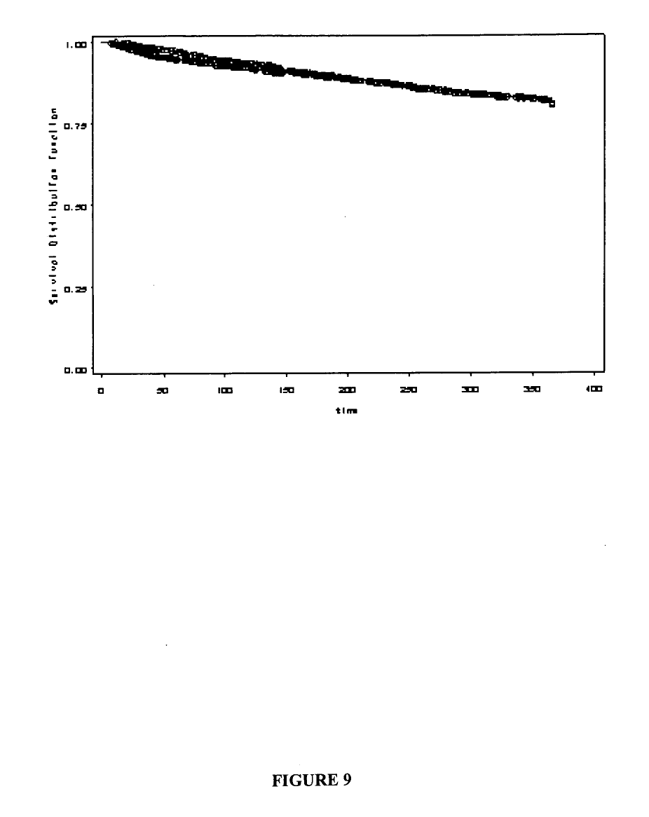

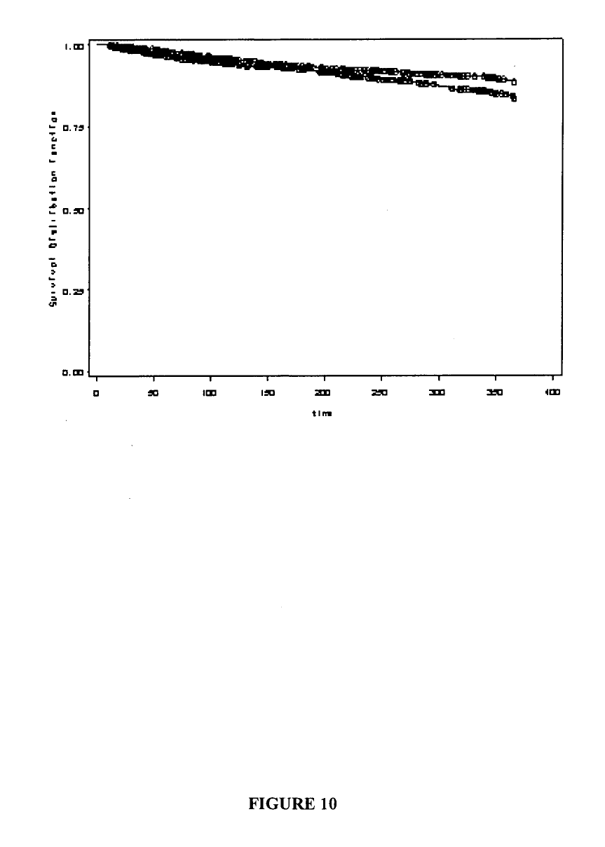

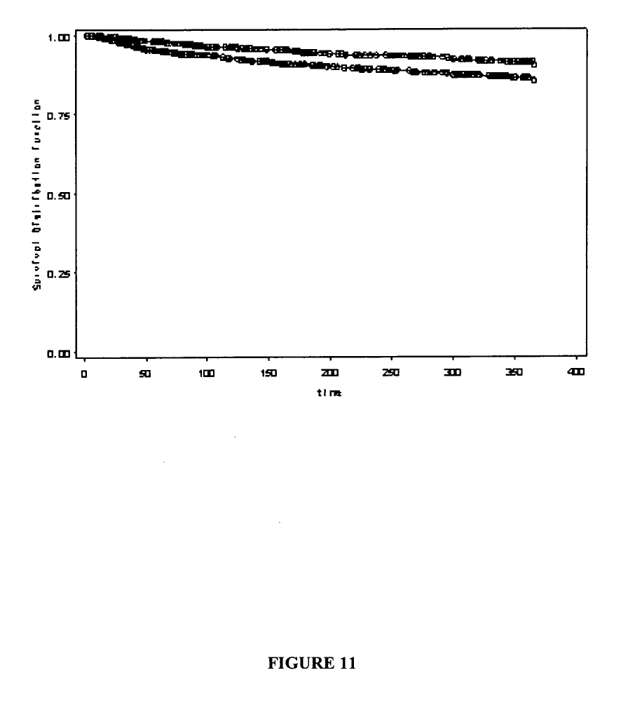

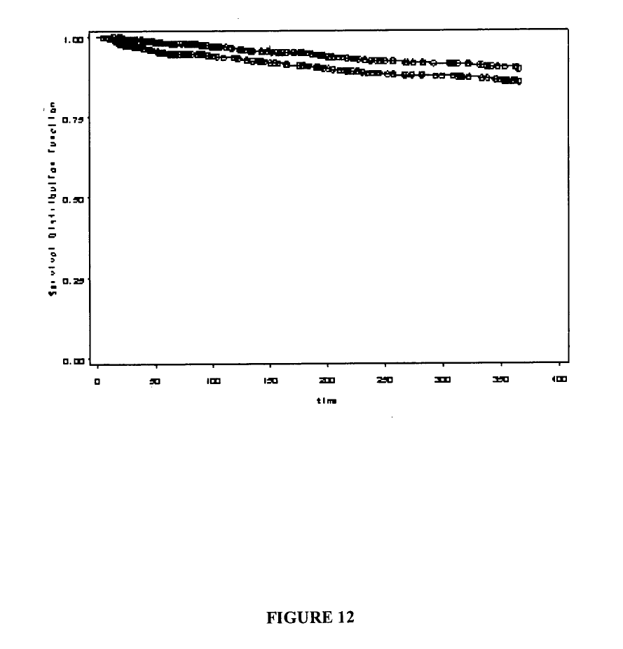

For all subjects in the study phosphate binder therapy improved one year survival rates. For example, a hazards ratio (HR) was calculated as 0.75 (95% CI 0.66-0.86; p<0.0001). See, FIG. 8. For low range normophophatemic patients selected with a serum phosphate <3.8 mg/dl (binder users, n=795; non-users, n=808) an HR of 0.99 was calculated (95% CI 0.78-1.25; p=0.9). See, FIG. 9. For high range normophosphatemic patients selected with a serum phosphate of between 3.8-4.7 mg/dl (binder users, n=792; non-users, n=8820 an HR of 0.70 was calculated (95% CI 0.52-0.92; p=0.01). See, FIG. 10. For patients selected with a slightly elevated serum phosphate between 4.8-5.5 mg/dl (binder users, n=885; non-users, n=952) an HR of 0.62 was calculated (95% CI 0.47-0.83; p=0.001). See, FIG. 11. For all other patients selected with an elevated serum phosphate of >5.5 mg/dl (binder users, n=861; non-users, n=714) an HR of 0.68 was calculated (95% CI 0.50-0.92; p=0.01). See, FIG. 12.

It should be noted that the estimate of the magnitude of the benefit of phosphorus binders on survival was not appreciably altered when comparing the overall unadjusted results (HR=0.75; 95% CI 0.66-0.86) to a model that adjusted for follow-up phosphate levels (HR=0.77; 95% CI 0.67-0.88; p=0.0001)(data not shown).

### CONCLUSION

These data demonstrate that early treatment (i.e., before the development of hyperphosphatemia) with dietary phosphorus binders is independently associated with decreased one-year mortality among incident dialysis patients. The benefit of phosphate binders was independent of baseline serum phosphate levels, spanned much of the normal range of serum phosphate, and was independent of follow-up serum phosphate levels suggesting potential benefits beyond reduction of serum phosphate.

### REFERENCES

60. Gutierrez O, Isakova T, Rhee E, et al. Fibroblast growth factor-23 mitigates hyperphosphatemia but accentuates calcitriol deficiency in chronic kidney disease. J Am Soc Nephrol. 2005; 16:2205-15.
61. Larsson T, Nisbeth U, Ljunggren O, Juppner H, Jonsson K B. Circulating concentration of FGF-23 increases as renal function declines in patients with chronic kidney disease, but does not change in response to variation in phosphate intake in healthy volunteers. Kidney Int. 2003; 64:2272-9.
62. Shigematsu T, Kazama J J, Yamashita T, et al. Possible involvement of circulating fibroblast growth factor 23 in the development of secondary hyperparathyroidism associated with renal insufficiency. Am J Kidney Dis. 2004; 44:250-6.
63. Block G A, Klassen P S, Lazarus J M, Ofsthun N, Lowrie E G, Chertow G M. Mineral metabolism, mortality, and morbidity in maintenance hemodialysis. J Am Soc Nephrol 2004; 15:2208-18.
64. Young E W, Akiba T, Albert J M, et al. Magnitude and impact of abnormal mineral metabolism in hemodialysis patients in the Dialysis Outcomes and Practice Patterns Study (DOPPS). Am J Kidney Dis. 2004; 44:34-8.
65. K/DOQI clinical practice guidelines for bone metabolism and disease in chronic kidney disease. Am J Kidney Dis. 2003; 42:1-201.
66. Westerberg P A, Linde T, Wikstrom B, Ljunggren O, Stridsberg M, Larsson T E. Regulation of fibroblast growth factor-23 in chronic kidney disease. Nephrol Dial Transplant. 2007; In Press.
67. Liu S, Quarles L D. How fibroblast growth factor 23 works. J Am Soc Nephrol. 2007; 18:1637-47.
68. Isakova T, Gutierrez O, Shah A, et al. Postprandial calcium metabolism and secondary hyperparathyroidism in early chronic kidney disease. J Am Soc Nephrol. 2007; In Press.
69. Schwarz S, Trivedi B K, Kalantar-Zadeh K, Kovesdy C P. Association of disorders in mineral metabolism with progression of chronic kidney disease. Clin J Am Soc Nephrol. 2006; 1:825-31.
70. Voormolen N, Noordzij M, Grootendorst D C, et al. High plasma phosphate as a risk factor for decline in renal function and mortality in pre-dialysis patients. Nephrol Dial Transplant. 2007.
71. Norris K C, Greene T, Kopple J, et al. Baseline predictors of renal disease progression in the African American Study of Hypertension and Kidney Disease. J Am Soc Nephrol. 2006; 17:2928-36.
72. Haut L L, Alfrey A C, Guggenheim S, Buddington B, Schrier N. Renal toxicity of phosphate in rats. Kidney Int. 1980; 17:722-31.
73. Barsotti G, Giannoni A, Morelli E, et al. The decline of renal function slowed by very low phosphorus intake in chronic renal patients following a low nitrogen diet. Clin Nephrol. 1984; 21:54-9.
74. Fliser D, Kollerits B, Neyer U, et al. Fibroblast Growth Factor 23 (FGF23) Predicts Progression of Chronic Kidney Disease: The Mild to Moderate Kidney Disease (MMKD) Study. J Am Soc Nephrol. 2007; 18:26,00-8.
75. Block G A, Hulbert-Shearon T E, Levin N W, Port F K. Association of serum phosphorus and calcium x phosphate product with mortality risk in chronic hemodialysis patients: a national study. Am J Kidney Dis. 1998; 31:607-17.
76. Ganesh S K, Stack A G, Levin N W, Hulbert-Shearon T, Port F K. Association of elevated serum PO(4), Ca×PO(4) product, and parathyroid hormone with cardiac mortality risk in chronic hemodialysis patients. J Am Soc Nephrol. 2001; 12:2131-8.
77. Urakawa I, Yamazaki Y, Shimada T, et al. Klotho converts canonical FGF receptor into a specific receptor for FGF23. Nature. 2006; 444:770-4.
78. Yu X, Ibrahimi O A, Goetz R, et al. Analysis of the biochemical mechanisms for the endocrine actions of fibroblast growth factor-23. Endocrinology. 2005; 146:4647-56.
79. Koiwa F, Kazama J J, Tokumoto A, et al. Sevelamer hydrochloride and calcium bicarbonate reduce serum fibroblast growth factor 23 levels in dialysis patients. Ther Apher Dial. 2005; 9:336-9.
80. Nagano N, Miyata S, Abe M, et al. Effect of manipulating serum phosphorus with phosphate binder on circulating PTH and FGF23 in renal failure rats. Kidney Int. 2006; 69:531-7.
81. Levin A, Bakris G L, Molitch M, et al. Prevalence of abnormal serum vitamin D, PTH, calcium, and phosphorus in patients with chronic kidney disease: results of the study to evaluate early kidney disease. Kidney Int. 2007; 71:31-8.
82. McClellan W, Warnock D G, McClure L, et al. Racial differences in the prevalence of chronic kidney disease among participants in the Reasons for Geographic and Racial Differences in Stroke (REGARDS) Cohort Study. J Am Soc Nephrol. 2006; 17:1710-5.
83. Bell N H, Greene A, Epstein S, Oexmann M J, Shaw S, Shary J. Evidence for alteration of the vitamin D-endocrine system in blacks. J Clin Invest. 1985; 76:470-3.
84. Bhan I, Shah A, Holmes J, et al. Post-transplant hypophosphatemia: Tertiary ‘Hyper-Phosphatoninism’? Kidney hit. 2006; 70:1486-94.
85. Ambuhl P M, Meier D, Wolf B, Dydak U, Boesiger P, Binswanger U. Metabolic aspects of phosphate replacement therapy for hypophosphatemia after renal transplantation: impact on muscular phosphate content, mineral metabolism, and acid/base homeostasis. Am J Kidney Dis. 1999; 34:875-83.
86. Levi M. Post-transplant hypophosphatemia. Kidney Int. 2001 59:2377-87.
87. Rosenbaum R W, Hruska K A, Korkor A, Anderson C, Slatopolsky E. Decreased phosphate reabsorption after renal transplantation: Evidence for a mechanism independent of calcium and parathyroid hormone. Kidney hit. 1981; 19:568-78.
88. Felsenfeld et al., Hypophosphatemia in long-term renal transplant recipients: effects on bone histology and 1,25-dihydroxycholecalciferol. Min Electrol Metabol. 1986; 12:333-41.
89. Green J, Debby H, Lederer E, Levi M, Zajicek H K, Bick T. Evidence for a PTH-independent humoral mechanism in post-transplant hypophosphatemia and phosphaturia. Kidney Int. 2001; 60:1182-96.
90. Riancho J A, de Francisco A L, del Arco C, et al. Serum levels of 1,25-dihydroxyvitamin D after renal transplantation. Min Electrol Metabol 1988; 14:332-7.
91. Steiner R W, Ziegler M, Halasz N A, Catherwood B D, Manolagas S, Deftos L J. Effect of daily oral vitamin D and calcium therapy, hypophosphatemia, and endogenous 1-25 dihydroxycholecalciferol on parathyroid hormone and phosphate wasting in renal transplant recipients. Transplantation. 1993; 56:843-6.
92. Wolf M, Ketttle E, Sandier L, Ecker J L, Roberts J, Thadhani R, Obesity and preeclampsia: the potential role of inflammation. Obstet GynecoL 2001; 98:757-62.
93. Wolf M, Sandier L, Jimenez-Kimble R, Shah A, Ecker J L, Thadhani R. Insulin resistance but not inflammation is associated with gestational hypertension. Hypertension. 2002; 40:886-91.
94. Wolf M, Sandller L, Munoz K, Hsu K, Ecker J L, Thadhani R. First trimester insulin resistance and subsequent preeclampsia: a prospective study. J Clin Endocrinol Metab. 2002; 87:1563-8.
95. Wolf M, Sandier L, Hsu K, Vossen-Smirnakis K, Ecker J L, Thadhani R. First-trimester C-reactive protein and subsequent gestational diabetes. Diabetes Care. 2003; 26:819-24.
96. Wolf M, Sauk J, Shah A, et al. Inflammation and glucose intolerance: a prospective study of gestational diabetes mellitus. Diabetes Care. 2004; 27:21-7.
97. Wolf M, Shah A, Jimenez-Kimble R, Sauk J, Ecker J L, Thadhani R. Differential risk of hypertensive disorders of pregnancy among Hispanic women. J Am Soc Nephro/. 2004; 15:1330-8.
98. Wolf M, Shah A, Lam C, et al. Circulating levels of the antiangiogenic marker sFLT-1 are increased in first versus second pregnancies. Am J Obstet Gyneco/. 2005; 193:16-22.
99. Wolf M, Hub,el C A, Lain C, et al. Preeclampsia and future cardiovascular disease: potential role of altered angiogenesis and insulin resistance. J C/in Endocrino/Metab. 2004; 89:6239-43.
100. Teng M, Wolf M, Lowrie E, Ofsthun N, Lazarus J M, Thadhani R. Survival of patients undergoing hemodialysis with paricalcitol or calcitriol therapy. N Engl J Meal. 2003; 349:446-56.
101. Teng M, Wolf M, Ofsthun M N, et al. Activated injectable vitamin D and hemodialysis survival: a historical cohort study. J Am Soc Nephro/. 2005; 16:1115-25.
102. Wolf M, Shah A, Gutierrez O, et al. Vitamin D levels and early mortality among incident hemodialysis patients. Kidney Int. 2007; In Press.
103. Giovannucci E, Liu Y, Rimm E S, et al. Prospective study of predictors of vitamin D status and cancer incidence and mortality in men. J Natl Cancer Inst. 2006; 98:451-9.
104. Hypponen E, Laara E, Reunanen A, Jarvelin M R, Virtanen S M. Intake of vitamin D and risk of type 1 diabetes: a birth-cohort study. Lancet. 2001; 358:1500-3.
105. Li Y C, Kong J, Wei M, Chen Z F, Liu S Q, Cao L P. 1,25-Dihydroxyvitamin D(3) is a negative endocrine regulator of the renin-angiotensin system. J C/in Invest. 2002; 110:229-38.
106. Munger K L, Zhang S M, O'Reilly E, et al. Vitamin D intake and incidence of multiple sclerosis. Neurology. 2004; 62:60-5.
107. Pittas A G, Dawson-Hughes B, Li T, et al. Vitamin D and calcium intake in relation to type 2 diabetes in women. Diabetes Care. 2006; 29:650-6.
108. Wu J, Garami M, Cao L, Li Q, Gardner D G. 1,25(OH)2D3 suppresses expression and secretion of atrial natriuretic peptide from cardiac myocytes. Am J Physiol. 1995; 268:E1108-13.
109. Xiang W, Kong J, Chen S, et al. Cardiac hypertrophy in vitamin D receptor knockout mice: role of the systemic and cardiac renin-angiotensin systems. Am J Physiol Endocrinol Metab. 2005; 288:E125-32.
110. Zittermann A. Vitamin D and disease prevention with special reference to cardiovascular disease. Prog Biophys Mol Biol. 2006.
111. Foley R N, Parfrey P S, Sarnak M J, Clinical epidemiology of cardiovascular disease in chronic renal disease. Am J Kidney Dis. 1998; 32:112-9.
112. LaClair R E, Hellman R N, Karp S L, et al. Prevalence of calcitriol deficiency in CKD: a cross-sectional study across latitudes in the United States. Am J Kidney Dis. 2005; 45:1026-33.
113. Kalantar-Zadeh K, Kuwae N, Regidor D L, et al. Survival predictability of time-varying indicators of bone disease in maintenance hemodialysis patients. Kidney Int. 2006; 70:771-80.
114. Melamed M L, Eustace J A, Plantinga L, et al. Changes in serum calcium, phosphate, and PTH and the risk of death in incident dialysis patients: a longitudinal study. Kidney Int. 2006; 70:351-7.
115. Bloembergen W E, Port F K, Mauger E A, Wolfe R A. Causes of death in dialysis patients: racial and gender differences. J Am Soc Nephrol. 1994; 5:1231-1242.
116. Frankenfield D L, Rocco M V, Roman S H, McClellan W M. Survival advantage for adult Hispanic hemodialysis patients? Findings from the end-stage renal disease clinical performance measures project. J Am Soc Nephrol. 2003; 14:180-6.
117. Robinson B M, Joffe M M, Pisoni R L, Port F K, Feldman H I. Revisiting survival differences by race and ethnicity among hemodialysis patients: the Dialysis Outcomes and Practice Patterns Study. J Am Soc Nephrol. 2006; 17:2910-8.
118. USRDS: the United States Renal Data System. Am J Kidney Dis. 2003; 42:1-230.
119. Foley R N, Collins A J. End-Stage Renal Disease in the United States: An Update from the United States Renal Data System. J Am Soc Nephrol. 2007; In Press.
120 Bringhurst F, Demay M, Kronenberg H. Hormones and disorders of mineral metabolism. In: Larsen P, Kronenbeq; 1 H, Melmed S, Polonsky K, eds. Williams Textbook of Endocrinology. 10th ed. Philadelphia: Saunders, An Imprint of Elsevier Science 2003:1303-1371.
121. Agatston A S, Janowitz W R, Hildner F J, Zusmer N R, Viamonte M, Jr., Detrano R. Quantification of coronary artery calcium using ultrafast computed tomography. J Am Coil Cardiol. 1990; 15:827-32.
122. Verdecchia P, Schillaci G, Borgioni C, et al. Prognostic value of left ventricular mass and geometry in systemic hypertension with left ventricular hypertrophy. Am J Cardiol. 1996; 78:197-202.
123. Gutierrez O, Isakova T, Andress D L, Levin A, Wolf M. Prevalence and severity of disordered mineral metabolism in Blacks with chronic kidney disease. Kidney Int. 2007; In press.
124. Gupta A, Kallenbach L R, Zasuwa G, Divine G W. Race is a major determinant of secondary hyperparathyroidism in uremic patients. J Am Soc Nephrol. 2000; 11:330-4.
125. Feldman H I, Appel L J, Chertow G M, et al. The Chronic Renal Insufficiency Cohort (CRIC) Study: Design and Methods. J Am Soc Nephrol. 2003; 14:S148-53.
126. Levey A S, Greene T, Schluchter M D, et al. Glomerular filtration rate measurements in clinical trials. Modification of Diet in Renal Disease Study Group and the Diabetes Control and Complications Trial Research Group. J Am Soc Nephrol. 1993; 4:1159-71.
127. Perrone R D, Steinman T I, Beck G J, et al. Utility of radioisotopic filtration markers in chronic renal insufficiency: simultaneous comparison of ¹²⁵I-iothalamate, ¹⁶⁹Yb-DTPA, ⁹⁹mTc-DTPA, and inulin. The Modification of Diet in Renal Disease Study. Am J Kidney Dis. 1990; 16:224-35.
128. K/DOQI Clinical Practice Guidelines for Evaluation of Chronic Kidney Disease: Evaluation, Classification., and Stratification. Am J Kidney Dis. 2002; 39:S46-S76.
129. Belch et al., Critical issues in Peripheral Arterial Disease Detection and Management: A Call to Action. Arch Intern Med. 2003; 163:884-892.
130. London G M, Blacher J, Pannier B, Guerin A P, Marchais S J, Safar M E. Arterial wave reflections and survival in end-stage renal failure. Hypertension. 2001; 38:434-8.
131. Blacher J, Guerin A P, Pannier B, Marchais S J, Safar M E, London G M. Impact of aortic stiffness on survival in end-stage renal disease. Circu/ation. 1999; 99:2434-9.
132. London G M, Marchais S J, Safar M E, et al. Aortic and large artery compliance in end-stage renal failure. Kidney Int. 1990; 37:137-42.
133. Brenner B M, Cooper M E, de Zeeuw D, et al. Effects of losartan on renal and cardiovascular outcomes in patients with type 2 diabetes and nephropathy. N Engl J Med. 2001; 345:861-9.
134. Greaves S C, Gamble G D, Collins J F, Whalley C A, Sharpe D N. Determinants of left ventricular hypertrophy and systolic dysfunction in chronic renal failure. Am J Kidney Dis. 1994; 24:768-76.
135. Levin et al., Prevalent left ventricular hypertrophy in the predialysis population: identifying opportunities for intervention. Am J Kidney Dis. 1996; 27:347-54.
136. Levin A, Thompson O R, Ethier J, et al. Left ventricular mass index increase in early renal disease: impact of decline in hemoglobin. Am J Kidney Dis. 1999; 34:125-34.
137. Ernesto P, Diego B, Paolo C, Davide R, Giuseppe C. Left Ventricular Hypertrophy in Nondiabetic Predialysis CKD. Am J Kidney Dis. 2005; 46:320-327.
138. Braun J, Oldendorf M, Moshage W, Heidler R, Zeitler E, Luft F C. Electron beam computed tomography in the evaluation of cardiac calcification in chronic dialysis patients. Am J Kidney Dis. 1996; 27:394-401.
139. Block G A, Raggi P, Bellasi A, Kooienga L, Spiegel D M. Mortality effect of coronary calcification and phosphate binder choice in incident hemodialysis patients. Kidney Int. 2007; 71:438-41.
140. Oh et al., Advanced Coronary and Carotid Arteriopathy in Young Adults With Childhood-Onset Chronic Renal Failure. Circulation. 2002; 106:100-105.
141. Raggi P, Boulay A, Chasan-Taber S, et al. Cardiac calcification in adult hemodialysis patients: A link between end-stage renal disease and cardiovascular disease? J Am Coll Cardiol. 2002; 39:695-701.
142. Chertow G M, Burke S K, Raggi P. Sevelamer attenuates the progression of coronary and aortic calcification in hemodialysis patients. Kidney Int. 2002; 62:245-52.
143. Block C A, Spiegel D M, Ehrlich J, et al. Effects of sevelamer and calcium on coronary artery calcification in patients new to hemodialysis. Kidney Int. 2005; 68:1815-24.
144. Arad Y, Spadaro L A, Goodman K, et al. Predictive value of electron beam computed tomography of the coronary arteries. 19-month follow-up of 1173 asymptomatic subjects. Circulation. 1996; 93:1951-3.
145. Haydar A A, Hujairi N M, Covic A A, Pereira D, Rubens M, Goldsmith D J. Coronary artery calcification is related to coronary atherosclerosis in chronic renal disease patients: a study comparing EBCT-generated coronary artery calcium scores and coronary angiography. Nephrol Dial Transplant. 2004; 19:2307-12.
146. Greenland P., Bonow R O, Brundage B H, et al. ACCF/AHA 2007 Clinical Expert Consensus Document on Coronary Artery Calcium Scoring By Computed Tomography in Global Cardiovascular Risk Assessment and in Evaluation of Patients With Chest Pain: A Report of the American College of Cardiology Foundation Clinical Expert Consensus Task Force (ACCF/AHA Writing Committee to Update the 2000 Expert Consensus Document on Electron Beam Computed Tomography) Developed in Collaboration With the Society of Atherosclerosis Imaging and Prevention and the Society of Cardiovascular Computed Tomography. J Am Coll Cardiol. 2007; 49:378-402.
147. Budoff M J, Achenbach S, Blumenthal R S, et al. Assessment of Coronary Artery Disease by Cardiac Computed Tomography: A Scientific Statement From the American Heart Association Committee on Cardiovascular Imaging and Intervention, Council on Cardiovascular Radiology and Intervention, and Committee on Cardiac Imaging, Council on Clinical Cardiology. Circulation. 2006; 114:1761-1791.
148. Budoff M J, Diamond G A, Raggi P, et al. Continuous Probabilistic Prediction of Angiographically Significant Coronary Artery Disease Using Electron Beam Tomography. Circulation. 2002; 105:1791-1796.
149. Haberl R, Becket A, Leber A, et al. Correlation of coronary calcification and angiographically documented stenoses in patients with suspected coronary artery disease: results of 1,764 patients. J Am Coil Cardiol. 2001; 37:451-457.
150. Klassen P S, Lowrie E G, Reddan D N, et al. Association between pulse pressure and mortality in patients undergoing maintenance hemodialysis. JAMA. 2002; 287:1548-55.
151. Newman A B, Sutton-Tyrrell K, Vogt M T, Kuller L H. Morbidity and mortality in hypertensive adults with a low ankle/arm blood pressure index. JAMA. 1993; 270:487-9.
152. Weitz J I, Byrne J, Clagett G P, et al. Diagnosis and Treatment of Chronic Arterial Insufficiency of the Lower Extremities: A Critical Review. Circulation. 1996; 94:3026-3049.
153. Guerrero A, Montes R, Munoz-Terol J, et al. Peripheral arterial disease in patients with stages IV and V chronic renal failure. NephroL Dial. Transplant. 2006; 21:3525-3531.
154. Yrjö L, Juha P S, Terho L, Heini H, Heikki S. The prevalence of peripheral arterial disease and medial arterial calcification in patients with chronic renal failure: Requirements for diagnostics. Am J Kidney Dis. 2002; 40:472-479.
155. de Vinuesa S G, Ortega M, Martinez P, Goicoechea M, Campdera F G, Luno J. Subclinical peripheral arterial disease in patients with chronic kidney disease: Prevalence and related risk factors. Kidney Int. 2005; 67:S44.-S47.
156. Ono K, Tsuchida A, Kawai H, et al. Ankle-Brachial Blood Pressure Index Predicts All-Cause and CardiovascuLar Mortality in Hemodialysis Patients. J Am Soc Nephrol 2003; 14:1591-1598.
157. Suki W N, Zabaneh R, Cangiano J L, et al. Effects of sevelamer and calcium-based phosphate binders on mortality in hemodialysis patients. Kidney Int. 2007.
158. Rothman K J, Greenland S. Modem Epidemeology. 2nd ed Boston: Little, Brown; 1998.
159. Tebben P J, Kalli K R, Cliby W A, et al. Elevated fibroblast growth factor 23 in women with malignant ovarian tumors. Mayo Clin Proc. 2005; 80:745-51.
160. Walton R J, Bijvoet O L. Nomogram for derivation of renal threshold phosphate concentration. Lancet. 1975; 2:309-310.
161. Grunkemeier G L, Anderson R P, Miller D C, Starr A. Time-related analysis of nonfatal heart valve complications;: cumulative incidence (actual) versus Kaplan-Meier (actuarial). Circulation. 1997; 96:11-70-4; discussion 11-74-5.
162. Grunkemeier G L, Jamieson W R, Miller D C, Starr A. Actuarial versus actual risk of porcine structural valve deterioration. J Thorac Cardiovasc Surg. 1994; 108:709-18.
163. Kleinbaum D G, Kupper L, Muller K E, Nizam A. Applied Regression Analysis and Other Multivariable Methods. 3rd ed Pacific Grove: Duxbury Press; 1998.
164. Imanishi Y, Inaba M, Nakatsuka K, et al. FGF-23 in patients with end-stage renal disease on hemodialysis. Kidney Int. 2004; 65:1943-6.
165. Carpenter T O, Ellis B K, Insogna K L, Phiibrick W M, Sterpka J, Shimkets R. Fibroblast growth factor 7: an inhibitor of phosphate transport derived from oncogenic osteomalacia-causing tumors. J Clin Endocrinol Metab. 2005; 90:1012-20.
166. Kumar R. New insights into phosphate homeostasis: fibroblast growth factor 23 and frizzled-related protein-4 are phosphatudc factors derived from tumors associated with osteomalacia. Curr Opin Nephrol Hypertens. 2002; 11:547-53.
167. Razzaque M S, Sitara D, Taguchi T, St-Arnaud R, Lanske B. Premature aging-like phenotype in fibroblast growth factor 23 null mice is a vitamin D-mediated process. Faseb J. 2006; 20:720-2.
168. Kuro-o M, Matsumura Y, Aizawa H, et al. Mutation of the mouse klotho gene leads to a syndrome resembling ageing. Nature. 1997; 390:45-51.

I claim:

1-8. (canceled)

9. A method, comprising:
a) providing:
i) an asymptomatic patient having a mortality risk due to chronic kidney disease;
ii) a biological sample comprising an amino acid sequence derived from an FGF-23 protein;
b) detecting said amino acid sequence having an activity ranging between approximately 30-60 RU/ml; and,
c) assigning a mortality risk by comparing said activity by comparing said activity to a reference population.

10. The method of claim 9, wherein said mortality risk is two times that of said reference population.

11. The method of claim 9, wherein said mortality risk is three times that of the reference population.

12. The method of claim 9, wherein said mortality risk is four times that of the reference population.

13. The method of claim 9, wherein said mortality risk is five times that of the reference population.

14. The method of claim 9, wherein said detecting comprises an antibody directed towards said amino acid sequence.

15. The method of claim 9, wherein said patient has a glomerular filtration rate of at least 90 ml/minute.

16-24. (canceled)
